(19) United States
(12) Patent Application Publication
(10) Pub. No.: US 2013/0142847 A1
(43) Pub. Date: Jun. 6, 2013

(54) S-ADENOSYLMETHIONINE FORMULATIONS WITH ENHANCED BIOAVAILABILITY

(71) Applicants: I. David MacDonald, Surrey (CA); Nancy Harrison, North Vancouver (CA); Aniko Takacs-Cox, North Vancouver (CA); Admir Purac, Burnaby (CA); Almira Blazek-Welsh, Vancouver (CA)

(72) Inventors: I. David MacDonald, Surrey (CA); Nancy Harrison, North Vancouver (CA); Aniko Takacs-Cox, North Vancouver (CA); Admir Purac, Burnaby (CA); Almira Blazek-Welsh, Vancouver (CA)

(73) Assignee: Methylation Sciences, Inc., Burnaby, BC (CA)

(21) Appl. No.: 13/811,772

(22) PCT Filed: Jul. 27, 2011
(86) PCT No.: PCT/CA2011/050462

§ 371 Date: Feb. 21, 2013

Related U.S. Application Data
(63) Continuation of application No. 12/845,555, filed on Jul. 28, 2010.
(60) Provisional application No. 61/435,997, filed on Jan. 25, 2011.
(60) Provisional application No. 61/229,194, filed on Jul. 28, 2009.

(51) Int. Cl. A61K 31/7076

(52) U.S. Cl. CPC A61K 31/7076
USPC 424/400; 514/46

(57) ABSTRACT

Provided herein are compositions and methods to enhance the absorption of S-adenosylmethionine (SAMe) and methods of treating various disorders or diseases using non-parenteral SAMe formulations with enhanced-absorption and improved bioavailability. In certain embodiments, the enhanced bioavailability formulations provided herein may be used to treat a variety of diseases or disorders, such as for example, psychiatric disorders including, generalized anxiety disorder, obsessive compulsive disorder, post traumatic stress disorder, panic disorder, depressive disorders (e.g. major clinical depression) and dysthymia; as well as treating liver disorders, cancer, autoimmune disorders, inflammatory disorders, joint disorders, gastrointestinal disorders and cardiovascular disease.

### CROSS-REFERENCE TO RELATED APPLICATIONS AND CLAIM TO PRIORITY

This application claims priority to U.S. Non-Provisional patent application Ser. No. 12/845,555, filed Jul. 28, 2010; and claims priority to U.S. Provisional patent application Ser. No. 61/435,997, filed Jan. 25, 2011 which are incorporated herein by reference in their entirety.

### TECHNICAL FIELD

Provided herein are compositions and methods for improved bioavailability of 5-adenosyl-L-methionine (“SAM-e” or “SAMe”). In certain embodiments, provided herein are formulations that modulate absorption of exogenous SAMe throughout the gastrointestinal tract and that provide, through oral administration or like method, a SAMe plasma concentration from which sufficient physiological effects can be expected. Provided herein are methods of treating a disease or disorder in a subject and/or improving the nutritional status of a subject by administering formulations enabling improved gastrointestinal absorption of SAMe, wherein increased gastrointestinal absorption is achieved using one or more absorption-enhancing technologies.

### BACKGROUND OF THE INVENTION

S-adenosyl-L-methionine (“SAM-e” or “SAMe”) is a naturally occurring compound that is present in tissues throughout the body. At the molecular level, SAMe is involved in various metabolic pathways, including transmethylation, transsulfuration and aminopropylation (e.g. in the production of polyamines, such as spermidine and spermine, from putrescine).

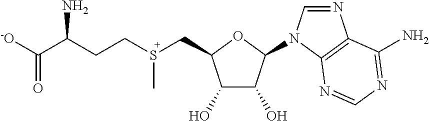

### S-adenosyl-L-methionine (SAMe)

In the body, SAMe is enzymatically synthesized from an amino acid, methionine, and a triphosphate nucleotide, ATP. SAMe has been tested in numerous clinical trials for the treatment of various ailments, including arthritis, liver disease and depression.

SAMe supplementation was initially considered impractical, due to the instability of the SAMe ion during manufacturing, shipping and storage. Eventually stable salts of SAMe were developed (such as SAMe tosylate disulfate, the butanedisulfonate salt of SAMe, the di-para-toluene sulfonate disulfate salt of SAMe, the tri-para-toluene sulfonic acid salt of SAMe and the like). These salts can be formulated using standard, known technologies used for non-parenteral administration including, but not limited to, tablets, capsules and pellets. Formulations such as these may also comprise a coating which can serve multiple purposes such as improving taste and ease of swallowing as well as reducing stomach irritation. Stable salts of SAMe are described in, for example, U.S. Pat. Nos. 3,954,726 and 4,057,686, both of which are incorporated herein by reference in their entirety. Conventional SAMe Active Pharmaceutical Ingredient (API) is supplied as a molecular entity comprising an ion along with several counter-ions. For example, SAMe ion plus a tosylate and 2 sulfonic acid counter-ions make up commercially available adenosylmethionine disulfate-p-toluenesulfonate (i.e. SAMe tosylate disulfate). When referring to SAMe dosing, it is currently accepted in the art that the numerical dose (usually in milligrams) refers to the amount of SAMe ion which is administered. For example, reference to a “400 mg SAMe tablet” using SAMe tosylate disulfate would include the 400 mg of SAMe ion, another 370 mg of the counter-ions, and 200-300 mg of additional excipient to make up a final tablet weight of 1.0-1.1 grams. Thus, for example, a 1600 mg oral dose of SAMe which is generally reported in the art would typically be a dose of four such 1.0-1.1 gram tablets taken at one time. Alternatively, the same 1600 mg dose of SAMe ion may also be accomplished by administration of other combinations of multiple tablets such as, sixteen 100 mg or eight 200 mg tablets of SAMe ion taken at a given time. Conventional oral dosage forms of SAMe are most commonly produced with about 400 mg of SAME ion; above that, the larger dosage form becomes difficult for swallowing considering that even at 400 mg of SAMe ion the tablets are quite large at 1.0-1.1 grams.

The prevailing conventional wisdom in the art is of the view that gastric juices in the stomach will alter the structure and/or function of SAMe thereby reducing its absorption and therefore a pH-specific coating which bypasses any SAMe release in the stomach is deemed necessary for oral administration. These “enteric” coatings are well known and routinely used by those of skill in the art as they are thought to provide a barrier which protects the encapsulated agent from the extremely low pH environment of the stomach. Although ‘pH-sensitive’, these coatings are designed solely to protect the encapsulated agent from the stomach. They generally begin to dissolve at a pH above about 5.5 (designed to match the pH of the environment immediately following the stomach) to allow release of the underlying dosage form. Various attempts to improve the stability and delivery of enteric coated SAMe have been reported. Rao et al., describe the use of an enteric coated, lipophilic soft gelatin capsule which begins dissolving at pH 5.5 (U.S. Pat. No. 6,759,395). They recommend the use of a lipophilic material to insulate SAMe salts as a means of protecting the encapsulated drug. Furthermore, they utilize a standard enteric coating in order to bypass any SAMe release in the stomach. The use of an enteric coating is not surprising; in view of prior art reports that SAMe cannot be absorbed in the stomach as it will be degraded first by gastric juices.

In addition to the reported claims in the art that SAMe is inactivated within the stomach, there is also a widely accepted belief in these arts surrounding the absorption mechanism and metabolism of this compound. Based on past clinical experimentation, SAMe is cited as being highly soluble and highly permeable yet exhibits low bioavailability. Studies using radiolabelled SAMe indicated that SAMe is readily absorbed within the gastrointestinal (GI) tract; however; plasma analysis showed very low bioavailability (Stramentinoli, G., (1987) The American Journal of Medicine 83(S 5A): 35-42). Therefore, those skilled in these arts assumed that SAMe's low bioavailability is caused by other factors, such as “first pass metabolism” in the liver. Over the past 20 years, numerous groups have attempted to understand SAMe bioavailability by looking at the pharmacokinetics, drug elimination and renal excretion profiles of various SAMe formulations but not the absorption mechanisms. It is routinely reported by those most knowledgeable in these arts that SAMe bioavailability when orally administered is limited to less than 5% because of “significant liver metabolism” prior to entering the blood (Bottiglieri et al., (1988) Alabama Journal of Medical Sciences 25(3): 296-301; Bottiglieri et al., (1997) Exp. Opin. Invest. Drugs 6(4): 417-426; Kaye et al., (1990) Drugs 40: 124-128). Additional drug elimination and renal excretion studies report that body accumulation of intact SAMe is unlikely as a cause of reduced bioavailability and instead also suggest that “active pre-systemic metabolism” is the cause (Giulidori and Cortellaro (1984) European Journal of Clinical Pharmacology 27: 119-121; Stramentinoli, G., (1986) Biological Methylation and Drug Design. R. T. Borchardt. New Jersey, Humana Press: 315-326). Another belief is that metabolism of SAMe occurs rapidly via transmethylation (and to a lesser extent, transsulfuration and aminoprophylation) pathways after non-parenteral administration. More specifically, the skilled practitioners of these arts proposed that the methyl group of SAMe is removed and incorporated into stable pools with low turnover rates, such as proteins and phospholipids (Bottiglieri (1997) supra; Stramentinoli (1987) supra), and therefore results in the very limited bioavailability of SAMe itself.

Active liver metabolism occurs with many drugs and typically causes a lower cap on their bioavailability as seen with SAMe. Also, there is a vast amount of clinical data reported in the art which supports ready absorption of SAMe. It has thus been the general dogma in the art that low SAMe bioavailability is due primarily to extensive first pass metabolism in the liver.

A recent report looking at SAMe uptake into cells in culture finds that SAMe is poorly transported through a monolayer of Caco-2 cells and poorly absorbed by cultured rat hepatocytes (McMillan et al., (2005) J. of Pharmacy and Pharmacology, 57:599). There remains still a need to identify both the reasons why exogenous SAMe bioavailability is low and ways in which to increase it in vivo particularly for use in treating disease and/or nutritional status.

### SUMMARY OF THE INVENTION

The present inventors have discovered that low permeability of SAMe is the primary reason why: 1) in vivo SAMe bioavailability is limited, 2) SAMe exhibits different absorption patterns in different regions of the GI tract and, 3) levels of SAMe metabolites are not significantly elevated after oral administration. Furthermore, the inventors also show for the first time that it is possible in vivo to improve the bioavailability of SAMe by administering exogenous SAMe in combination with one or more absorption-enhancing technologies, especially tight junction modulating agents. Provided herein are novel compositions and methods that enhance the gastrointestinal absorption of SAMe.

The exemplary embodiments provided herein relate to methods and compositions for enhancing the absorption of S-adenosyl-L-methionine (“SAMe”) or its stable salts as a means to increase SAMe bioavailability. Use of compositions and methods provided herein in vivo improves bioavailability of SAMe as compared to conventional non-parenteral dosage forms of SAMe which do not contain physiologically relevant levels of absorption enhancers.

In certain embodiments, provided herein are non-parenteral compositions of SAMe in combination with at least one absorption-enhancing technology. Without being limited to any particular theory, absorption-enhancing technologies which act to increase absorption of a physiologically acceptable dosage of SAMe may work in a number of ways including, for example, increasing SAMe residence time in the GI tract (therefore allowing more opportunity for uptake); delivering SAMe to regions of the GI tract that exhibit increased drug absorption; adding “absorption enhancers” which increase either transcellular or paracellular transport of drugs (including agents which directly affect tight junction opening or penetration); encapsulating SAMe in nanocarriers that deliver SAMe directly to cells; or a combination of any of such technologies which modulate absorption. An “absorption-enhancing technology” is therefore any excipient, device, mechanism, technique, method, treatment parameter or the like which either directly or indirectly affects the absorption or uptake of SAMe. Many of these technologies may be designed to exploit or optimize SAMe's inherent cationic nature at specific pH levels, for example, some may act to maintain SAMe in its cationic form which is more easily absorbed (e.g. in the presence of a buffer or buffering system). Accordingly, in certain embodiments, the compositions provided herein can be combined with unconventional factors, such as diet (amount and/or type of food and/or beverage), dosing schedule, the presence or absence of a coating (i.e. uncoated SAMe may be more efficiently absorbed) as a suitable means of altering SAMe absorption. In some embodiments, administration of absorption-enhancing technologies prior to SAMe administration may optimize SAMe uptake.

Site-specific delivery of SAMe to segments of the GI tract exhibiting enhanced-absorption (also known as “absorption windows”) may be achieved with the use of pH-dependent coatings which target SAMe release in pH-specific regions of the GI tract. As used herein, pH-dependent coatings include any coating which bypasses release of SAMe in the stomach, for example: delayed release coatings, extended release coatings, enteric coatings and the like.

Some exemplary embodiments relate to compositions comprising pH-dependent coatings, wherein the composition of the pH-dependent coating acts to release a physiologically acceptable dosage of SAMe in segment-specific areas of the GI tract. In some embodiments, pH-dependent coatings allow release of SAMe in several regions along the entire GI tract in order to affect the site-specific effect of SAMe uptake and bioavailability. In some embodiments, absorption of SAMe may occur throughout the entire length of the GI tract. By identifying regions with enhanced-absorption of SAMe, formulations targeted to these regions can be administered to ensure better control of SAMe absorption and bioavailability. In certain embodiments, pH-dependent coatings are not employed as simple enteric coatings applied to avoid degradation in the stomach. In certain embodiments, the pH-dependent coatings provided herein enable targeted delivery in the GI tract.

In some exemplary embodiments, pH-dependent coatings act to co-deliver SAMe and the absorption enhancer to the upper small intestine where SAMe is better absorbed. In other embodiments, the pH-dependent coating results in delivery of SAMe and the absorption enhancer to the large intestine and/or colorectal regions, where SAMe is very poorly absorbed. A recent report in the art shows the inhibitory effects of SAMe on colon and gastric cancers cell growth in vitro (Luo et al., (2010) Int. J. of Biological Sciences; 6(7); 784-795). The inventors discovered that improving absorption of SAMe in these areas, or targeting SAMe-containing dosage forms uncoating, release and dissolution in these areas, particularly within the colorectal region, may provide a novel means for delivering SAMe within close proximity to these types of tumors as opposed to merely systemic delivery. Thus, in some embodiments, the pH-dependent coating is configured to deliver SAMe directly to the colon and/or colorectal region, resulting in local and/or systemic delivery of SAMe to those parts of the intestinal tract.

Thus, certain exemplary embodiments also relate to methods for increasing the bioavailability of SAMe by delivering pH-dependent coated formulations of SAMe which act to release a physiologically acceptable dosage of SAMe and an absorption enhancer in site-specific or disease-specific regions of the GI tract.

In certain embodiments, site-specific delivery of SAMe to segments of the GI tract exhibiting enhanced-absorption may be achieved with the use of non-pH-dependent coatings which target SAMe release in regions of the GI tract by delaying release of SAMe in a non-pH-dependent manner, e.g. for a period of time necessary for the dosage form to pass through the GI tract to the target portion of the GI tract, such as the duodenum, ileum, jejunum, colon and/or colorectal part of the GI tract.

In certain embodiments, several methods provided herein affect a delayed release with non-pH dependent polymers. These include soluble or erodible barrier systems, enzymatically degraded barrier systems, rupturable coating systems, and plugged capsule systems among others. Materials that can be used to obtain a delay in release suitable for this component can be, but are not limited to, polyethylene glycol (PEG) with molecular weight above 4,000 daltons (Carbowax), Polyethylene Oxides (Polyox), waxes such as white wax or bees wax, paraffin, acrylic acid derivatives (Eudragit), propylene glycol, and ethylcellulose. In some embodiments, these materials can be present in the range of 0.5-40% (W/W) of this component.

In certain embodiments, “absorption enhancers”, which also is meant to include agents known as “penetration enhancers”, “permeability enhancers” and “promoters” act directly on specific aspects of the GI tract, such as paracellular transport, and affect the absorption rate of numerous drugs.

In certain embodiments, provided herein are compositions which make use of absorption enhancers to increase or promote absorption of a physiologically acceptable dosage of SAMe as a mechanism for increasing SAMe bioavailability.

Certain exemplary embodiments relate to absorption enhancers which directly modulate the activity of tight junctions. These are known as tight junction penetration agents or tight junction modulating agents or tight junction opening agents. Tight junctions are intercellular junctions between cells that control permeability between the cells. In this way, materials (e.g. APIs) cannot pass between cells but rather must be taken up by the cell and thus enables the cells to regulate what is allowed through. Tight junctions occur in many regions throughout the body including the mouth, small intestine, large intestine and colon and vary in density/tightness within different regions. Within the GI tract, tight junctions refer to the areas between adjacent endothelial cells and act to regulate the uptake of digested materials. Tight junctions are highly regulated and are one of the key elements that form the barrier between the luminal environment of the GI tract and the rest of the body.

Certain embodiments as exemplified herein also relate to compositions and methods which incorporate tight junction opening agents to increase or promote absorption of a physiologically acceptable dosage of SAMe.

Furthermore, the inventors show for the first time that, when studying SAMe absorption, a subset of high potency tight junction opening absorption enhancers exists and that identification of such high potency tight junction opening agents may be carried out using, for example, a Caco-2 cell model as described herein. Based on the concentration of tight junction modulator utilized as well as the increase in SAMe permeability achieved, suitable high potency tight junction modulators elicit at least a two-fold increase in SAMe uptake in vitro when used at low concentrations (i.e. about 2 mM or below). More preferably, at least a three-fold increase in SAMe uptake occurs at low concentrations of these tight junction opening agents and, even more preferably, a four-fold or greater increase is achieved. Thus, some embodiments as exemplified herein also relate to compositions which incorporate high potency tight junction opening agents to increase or promote absorption of a physiologically acceptable dosage of SAMe. Certain other embodiments as exemplified herein relate to compositions which incorporate SAMe as well as one or more high potency tight junction opening agents, wherein said tight junction opening agent yields enhanced absorption and causes minimal to no cell disruption as measured in an in vitro cell permeability test. In certain embodiments, provided herein are methods for screening high potency tight junction opening agents. In some embodiments, a method provided herein is an in vitro cell permeability test. In some embodiments, a method provided herein is a method for screening high potency tight junction opening agent using a Caco-2 cell model.

In certain embodiments, pharmaceutical, medicinal, veterinary or nutritional preparations used for administering a physiologically acceptable dosage of SAMe include conventional solid or semi-solid tablets, pills, granules and capsules as well as controlled-release technologies such as pH-sensitive drug targeting, timed-release technologies, osmotic pumps, layered tablets, multiparticle tablets, nanocarriers or their combinations. When referring to “medicinal” preparations, purposes or treatments they are meant to include “medical foods”. Medical foods are defined by the U.S. Food and Drug Administration as a food which is formulated to be consumed or administered enterally under the supervision of a physician and which is intended for the specific dietary management of a disease or condition for which distinctive nutritional requirements, based on recognized scientific principles, are established by medical evaluation.

Certain exemplary embodiments further relate to compositions for non-parenteral administration of SAMe wherein SAMe is formulated in a solid or semi-solid composition which comprises one or more absorption-enhancing technology. In certain embodiments, provided herein are methods of treatment wherein pharmaceutical, medicinal, veterinary or nutritional preparations of SAMe are administered in conjunction with one or more absorption-enhancing technology. In a preferred embodiment, said absorption-enhancing technology is co-administered with said pharmaceutical, medicinal, veterinary or nutritional preparations of SAMe, and, even more preferably, said absorption-enhancing technology is included in said pharmaceutical, medicinal, veterinary or nutritional preparations of SAMe.

In certain embodiments, absorption-enhancing technologies need not form part of the administered SAMe preparations and may be administered separately. Depending on their specific mechanism of action, the chosen absorption-enhancing technology may be utilized either immediately before, after or concurrent with the SAMe formulations. In some embodiments, provided herein are novel methods of treating a disease or disorder in a subject in need thereof, wherein said method comprises administering a physiologically effective dosage of SAMe in combination with one or more absorption-enhancing technologies. In a preferred embodiment, said one or more absorption-enhancing technologies is a tight junction modulating or opening agent.

Certain exemplary embodiments relate to methods for increasing the bioavailability of SAMe in a subject by delivering a composition for non-parenteral administration comprising SAMe and at least one absorption-enhancing technology, wherein said absorption-enhancing technology acts either directly or indirectly to increase the absorption of a physiologically acceptable dosage of SAMe.

In certain embodiments, diseases and/or disorders treatable with SAMe formulations provided herein are selected from the group consisting of, but not limited to, a mental or psychiatric disorder (e.g. psychotic/mood or non-psychotic mental disorders exemplified by depression and substance related disorders, respectively), a nervous system disease/disorder (e.g. a central nervous system disease exemplified by Alzheimer's), other neurological disease/disorders (e.g. headaches and sleep disorders), conditions associated with injury to the central nervous system, a liver disease/disorder (e.g. alcoholic liver disease), a cancer (e.g. solid and blood-borne cancers), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper- or hypo-methylation, a gastrointestinal disease/disorder, a cardiovascular disease/disorder, a metabolic disease/disorder (e.g. Type 2 diabetes) and a disorder induced in whole or in part by oxidative or free-radical damage. Additional embodiments relate to combinations of SAMe with one or more active ingredients that are commonly prescribed or used for treatment of and/or prophylaxis of various diseases or disorders in a subject.

In certain embodiments, provided herein is a pharmaceutical composition comprising a therapeutically effective dosage of S-adenosylmethionine and at least one zwitterionic surfactant. In some embodiments, said zwitterionic surfactant enhances systemic delivery of S-adenosylmethionine as measured by an increase in Cₘₐₓ and/or AUC compared to a control composition that lacks said zwitterionic surfactant. In some embodiments, the Cₘₐₓ and/or AUC of said composition is at least 140% of the Cₘₐₓ and/or AUC obtained with a control composition that lacks said zwitterionic surfactant.

In some embodiments, said composition is a non-parenteral composition. In some embodiments, the non-parenteral composition is an oral dosage composition, optionally formulated as a tablet, a capsule, or a multiparticulate formulation. In some embodiments, the non-parenteral composition is formulated as a dietary supplement or a medical food. In some embodiments, further comprising at least one detergent, surfactant, zwitterionic surfactant, unsaturated cyclic urea, fatty acid, fatty amine, alkane sulfonate, bile acid, organic acid, cyclodextrin, chelating agent, salt of any of the foregoing, or a combination thereof.

In some embodiments, said zwitterionic surfactant is an acyl carnitine. In some embodiments, said acyl carnitine is a palmitoyl carnitine, lauroyl carnitine, stearoyl carnitine, myristoyl carnitine, decanoyl carnitine, or a salt thereof. In some embodiments, said zwitterionic surfactant is a sulfobetaine. In some embodiments, said sulfobetaine is sulfobetaine-10, sulfobetaine-12, sulfobetaine-14, sulfobetaine-16, or sulfobetaine-18.

In some embodiments, at least a portion of the composition is formulated to dissolve in at least one of the stomach, duodenum, jejunum, ileum, large intestine, or colon. In some embodiments, the composition comprises a pH sensitive coating. In some embodiments, the compositions provided herein further comprise at least one delayed release component. In some embodiments, the delayed release component is a pH-triggered enteric coating. In some embodiments, the delayed release component is formulated to dissolve in at least one of the duodenum, jejunum, ileum, large intestine, or colon.

In some embodiments, S-adenosylmethionine and said zwitterionic surfactant are in a ratio of from 1:100,000 to 100,000:1, or 1:150 to 150:1, or 1:10 to 10:1 (weight:weight). In some embodiments, S-adenosylmethionine and said zwitterionic surfactant are in a ratio of 1:1 (weight:weight). In some embodiments, the composition comprises 50 to 3200 mg of S-adenosylmethionine. In some embodiments, the composition comprises 50 to 1600 mg of S-adenosylmethionine.

In some embodiments, the compositions provided herein further comprise at least one delayed release component. In some embodiments, the delayed release component is a pH-triggered enteric coating. In some embodiments, the delayed release component is formulated to dissolve in at least one of the duodenum, jejunum, ileum, large intestine, or colon.

In certain embodiments, provided herein are compositions for use in the treatment of a condition selected from the group consisting of a mental or psychiatric disorder, a nervous system disease or disorder, a neurological disease or disorders, a condition associated with injury to the central nervous system, a liver disease or disorder, a cancer, a joint disease or disorder, an inflammatory disease or disorder, an autoimmune disease or disorder, a degenerative disease or disorder, a soft-tissue disease or disorder, a pain disease or disorder, a genetic disorder related to hyper- or hypo-methylation, a gastrointestinal disease or disorder, a cardiovascular disease or disorder, and a disorder induced in whole or in part by oxidative or free-radical damage.

In certain embodiments, provided herein are methods for increasing the bioavailability of exogenous SAMe administered to a subject, said method comprising administering to the subject a non-parenteral composition comprising a therapeutically effective dosage of S-adenosylmethionine and at least one zwitterionic surfactant. In some embodiments, the composition is an oral dosage composition. In some embodiments, the composition is formulated as a dietary supplement or a medicinal food. In some embodiments, the compositions further comprise a detergent, a surfactant, a zwitterionic surfactant, an unsaturated cyclic urea, a fatty acid, a fatty amine, an alkane sulfonate, a bile acid, an organic acid, a cyclodextrin, a chelating agent, a salt of any of the foregoing, or a combination thereof. In some embodiments, at least a portion of the composition is formulated to dissolve in at least one of the stomach, duodenum, jejunum, ileum, large intestine, or colon. In some embodiments, the composition comprises a pH sensitive coating. In some embodiments, said zwitterionic surfactant is administered either before or after administration of the composition comprising the at least one physiologically effective dosage of S-adenosylmethionine.

In certain embodiments, provided herein are methods of treating a disorder selected from the group consisting of a mental or psychiatric disorder, a nervous system disease or disorder, a neurological disease or disorders, a condition associated with injury to the central nervous system, a liver disease or disorder, a cancer, a joint disease or disorder, an inflammatory disease or disorder, an autoimmune disease or disorder, a degenerative disease or disorder, a soft-tissue disease or disorder, a pain disease or disorder, a genetic disorder related to hyper- or hypo-methylation, a gastrointestinal disease or disorder, a cardiovascular disease or disorder, and a disorder induced in whole or in part by oxidative or free-radical damage, comprising administering to a patient in need thereof a composition provided herein.

In certain embodiments, provided herein is a non-parenteral composition comprising at least one physiologically effective dosage of S-adenosylmethionine in combination with at least one absorption-enhancing technology. In some embodiments, the absorption-enhancing technology is one of gastroretentive dosage adjuvants, gastrointestinal segment-specific delivery systems, chemically derived absorption enhancing agents, tight junction penetration agents, tight junction opening agents, nanocarriers, a diet regimen, and a dosing regimen. In some embodiments, the non-parenteral composition is an oral dosage composition. In some embodiments, the non-parenteral composition is incorporated in a dietary supplement or a medical food.

In some embodiments, the non-parenteral dosage composition comprises at least one physiologically effective dosage of S-adenosylmethionine in combination with at least one of a tight junction penetration agent and tight junction opening agent. In some embodiments, the at least one of a tight junction penetration agent and tight junction opening agent is selected from the group consisting of detergents, surfactants, zwitterionic surfactants, unsaturated cyclic ureas, fatty acids, fatty amines, alkane sulfonates, bile acids, organic acids, cyclodextrins, chelating agents, salts of any of the foregoing, and combinations thereof. In some embodiments, at least one of a tight junction penetrating agent and a tight junction opening agent includes a zwitterionic surfactant. In some embodiments, the zwitterionic surfactant is an acyl carnitine. In some embodiments, the zwitterionic surfactant is a sulfobetaine. In some embodiments, at least one of a tight junction penetrating agent and a tight junction opening agent includes a fatty acid or a salt thereof. In some embodiments, at least one of a tight junction penetrating agent and a tight junction opening agent includes a fatty amine or a salt thereof. In some embodiments, at least one of a tight junction penetrating agent and a tight junction opening agent includes a bile acid or a salt thereof. In some embodiments, at least one of a tight junction penetrating agent and a tight junction opening agent includes detergent, a surfactant, an unsaturated cyclic urea, and organic acid, a cyclodextrin, a chelating agent, a salt of any thereof, or a combination of two or more thereof.

In some embodiments, at least a portion of the composition is configured to dissolve in at least one of the stomach, duodenum, jejunum and ileum. In some embodiments, at least a portion of the composition is configured to dissolve in the large intestine or colon. In some embodiments, the composition incorporates a pH sensitive coating.

In certain embodiments, provided herein is a method for increasing the bioavailability of exogenous SAMe administered to a subject, said method comprising administering to the subject a non-parenteral composition comprising at least one physiologically effective dosage of S-adenosylmethionine in combination with at least one absorption-enhancing technology. In some embodiments, the absorption-enhancing technology is one of gastroretentive dosage adjuvants, gastrointestinal segment-specific delivery systems, chemically derived absorption enhancing agents, tight junction penetration agents, tight junction opening agents, nanocarriers, a diet regimen, and a dosing regimen. In some embodiments, the composition is an oral dosage composition. In some embodiments, the composition is incorporated in a dietary supplement or a medicinal food. In some embodiments, the composition comprises a physiologically effective dosage of S-adenosylmethionine in combination with at least one of a tight junction penetration agent and tight junction opening agent. In some embodiments, the composition comprises at least one of a tight junction penetration agent and tight junction opening agent is selected from the group consisting of detergents, surfactants, zwitterionic surfactants, unsaturated cyclic ureas, fatty acids, fatty amines, alkane sulfonates, bile acids, organic acids, cyclodextrins, chelating agents, salts of any of the foregoing, and combinations thereof. In some embodiments, the composition comprises at least one of a tight junction penetrating agent and a tight junction opening agent includes a zwitterionic surfactant. In some embodiments, the composition comprises at least one of a tight junction penetrating agent and a tight junction opening agent includes a fatty acid or a salt thereof. In some embodiments, at least one of a tight junction penetrating agent and a tight junction opening agent includes a fatty amine or a salt thereof. In some embodiments, at least one of a tight junction penetrating agent and a tight junction opening agent includes a bile acid or a salt thereof. In some embodiments, at least one of a tight junction penetrating agent and a tight junction opening agent includes detergent, a surfactant, an unsaturated cyclic urea, and organic acid, a cyclodextrin, a chelating agent, a salt of any thereof, or a combination of two or more thereof. In some embodiments, at least a portion of the composition is configured to dissolve in at least one of the stomach, duodenum, jejunum and ileum. In some embodiments, at least a portion of the composition is configured to dissolve in the large intestine or colon. In some embodiments, the composition incorporates a pH sensitive coating. In some embodiments, the absorption-enhancing technology is administered either before or after administration of the composition comprising the at least one physiologically effective dosage of S-adenosylmethionine.

In some embodiments, provided herein is a method of treating in a patient a disorder selected from the group consisting of a mental or psychiatric disorder (e.g. psychotic/mood or non-psychotic mental disorders exemplified by depression and substance related disorders, respectively), a nervous system disease/disorder (e.g. a central nervous system disease exemplified by Alzheimer's), other neurological disease/disorders (e.g. headaches and sleep disorders), conditions associated with injury to the central nervous system, a liver disease/disorder (e.g. alcoholic liver disease), a cancer (e.g. solid and blood-borne cancers), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper- or hypo-methylation, a gastrointestinal disease/disorder, a cardiovascular disease/disorder, and a disorder induced in whole or in part by oxidative or free-radical damage, comprising administering to the patient in need thereof a composition provided herein.

In some embodiments, provided herein is a solid oral composition containing a physiologically effective amount of S-adenosylmethionine and a tight junction opening absorption enhancer at a ratio of from 1:100,000 to 100,000:1 (weight:weight). In some embodiments, the S-adenosylmethionine and the absorption enhancer are in a ratio of from 1:150 to 150:1. In some embodiments, the 5-adenosylmethionine and the absorption enhancer are in a ratio of from 1:10 to 10:1. In some embodiments, the solid oral composition further comprises at least one delayed release component. In some embodiments, the delayed release component is a pH-triggered, enteric coating applied to the sold oral composition. In some embodiments, the delayed release component is such that the composition is configured to dissolve in at least one of the duodenum, jejunum, and ileum. In some embodiments, the delayed release component is such that the release is configured to dissolve in the large intestine or colon. In some embodiments, the tight junction opening absorption enhancer is a high potency absorption enhancer as determined in a Caco-2 monolayer permeability assay. In some embodiments, the tight junction opening absorption enhancer comprises one or more detergents, surfactants, zwitterionic surfactants, unsaturated cyclic ureas, fatty acids, fatty amines, alkane sulfonates, bile acids, organic acids, cyclodetrins, chelating agents, and combinations thereof. In some embodiments, the tight junction opening absorption enhancer comprises one or more zwitterionic surfactants. In some embodiments, the one or more zwitterionic surfactants includes a sulfobetaine. In some embodiments, the tight junction opening absorption enhancer includes a fatty amine or a salt thereof. In some embodiments, the tight junction opening absorption enhancer includes a bile acid or a salt thereof. In some embodiments, the tight junction opening absorption enhancer includes a detergent, a surfactant, an unsaturated cyclic urea, an organic acid, a cyclodextrin, a chelating agent, a salt of any thereof, or a combination of two or more thereof. In some embodiments, the tight junction opening absorption enhancer includes a long-chain acyl carnitine zwitterionic surfactant or a salt thereof. In some embodiments, the tight junction opening absorption enhancer includes one of the long-chain acyl carnitine zwitterionic surfactants selected from palmitoyl carnitine, lauroyl carnitine, stearoyl carnitine, myristoyl carnitine, and decanoyl carnitine or a salt thereof. In some embodiments, the tight junction opening absorption enhancer includes palmitoyl carnitine or a salt thereof. In some embodiments, the composition is in an oral dosage form. In some embodiments, the oral dosage form is a tablet, a capsule or a multiparticulate formulation. In some embodiments, the dosage form comprises about 50 to about 500 mg of S-adenosylmethionine.

In some embodiments, provided herein is a composition for the treatment of a condition amenable to treatment with S-adenosylmethionine.

In some embodiments, provided herein is a method of treating a condition amenable to treatment with S-adenosylmethionine, comprising administering to a patient a physiologically effective amount of a composition provided herein.

In some embodiments, provided herein is a pharmaceutical, solid oral composition containing a physiologically effective dose of S-adenosylmethionine and a tight junction opening absorption enhancer, wherein the enhancer enhances systemic delivery of S-adenosylmethionine as measured by an increase in Cmax and/or AUC compared to a control composition that lacks said enhancer. In some embodiments, the Cmax and/or AUC obtained with the dose of S-adenosylmethionine and a tight junction is at least 140% of the Cmax and/or AUC obtained with the control formulation or greater. In some embodiments, solid oral composition further comprises at least one delayed release component. In some embodiments, the delayed release component is a pH-triggered, enteric coating applied to the sold oral composition. In some embodiments, the delayed release component is such that the composition is configured to dissolve in at least one of the duodenum, jejunum, and ileum. In some embodiments, the delayed release component is such that the release is configured to dissolve in the large intestine or colon. In some embodiments, the tight junction opening absorption enhancer is a high potency absorption enhancer as determined in a Caco-2 monolayer permeability assay. In some embodiments, the tight junction opening absorption enhancer comprises one or more detergents, surfactants, zwitterionic surfactants, unsaturated cyclic ureas, fatty acids, fatty amines, alkane sulfonates, bile acids, organic acids, cyclodetrins, chelating agents, and combinations thereof. In some embodiments, the tight junction opening absorption enhancer comprises one or more zwitterionic surfactants. In some embodiments, the one or more zwitterionic surfactants includes a sulfobetaine. In some embodiments, the tight junction opening absorption enhancer includes a fatty amine or a salt thereof. In some embodiments, the tight junction opening absorption enhancer includes a bile acid or a salt thereof. In some embodiments, the tight junction opening absorption enhancer includes a detergent, a surfactant, an unsaturated cyclic urea, an organic acid, a cyclodextrin, a chelating agent, a salt of any thereof, or a combination of two or more thereof. In some embodiments, the tight junction opening absorption enhancer includes a long-chain acyl carnitine zwitterionic surfactant or a salt thereof. In some embodiments, the tight junction opening absorption enhancer includes one of the long-chain acyl carnitine zwitterionic surfactants selected from palmitoyl carnitine, lauroyl carnitine, stearoyl carnitine, myristoyl carnitine, and decanoyl carnitine or a salt thereof. In some embodiments, the tight junction opening absorption enhancer includes palmitoyl carnitine or a salt thereof. In some embodiments, the composition is in an oral dosage form. In some embodiments, the oral dosage form is a tablet, a capsule or a multiparticulate formulation. In some embodiments, the dosage form comprises about 50 to about 500 mg of S-adenosylmethionine.

### BRIEF DESCRIPTION OF THE DRAWINGS

FIG. 1A is a full-scale graph of the average plasma concentration of SAMe versus time of subjects who were administered 800 mg of one of three segment-specific SAMe formulations comprising coatings designed to release SAMe in the proximal GI tract (duodenum/jejunum; squares), in the distal GI tract (ileum/ascending colon; triangles) and metered throughout the entire GI tract (circles); and

FIG. 1B is a magnified view of the graph in FIG. 1A which better highlights the separation between the lower average plasma concentration curves;

FIG. 2 is a graph showing the permeability of SAMe across a monolayer of Caco-2 human colonic adenocarcinoma cells alone and in the presence of EDTA or in the absence of calcium. Propranolol is included as a high permeability control;

FIG. 3A is a graph showing the permeability of SAMe across a monolayer of Caco-2 human colonic adenocarcinoma cells alone and in the presence of various tight junction modulators; and

FIG. 3B is a graph showing the average plasma concentration of SAMe versus time when SAMe is delivered both in the presence and absence of the tight junction modulating agent, caprate. Animal subjects were administered either a single dose of SAMe disulfate tosylate in combination with inactive excipients and sodium caprate formulated in an enteric-coated capsule, or the control consisting of the identical formulation but without sodium caprate; and

FIG. 3C is a graph showing the average plasma concentration of SAMe versus time when SAMe is delivered both in the presence and absence of the tight junction modulating agent, palmitoyl-DL-carnitine chloride (PCC). Animal subjects were administered either a single dose of SAMe disulfate tosylate in combination with inactive excipients and PCC formulated in an enteric-coated capsule, or the control consisting of the identical formulation but without PCC.

FIG. 4A is a graph showing the average plasma concentration of SAMe versus time when SAMe is delivered both in the presence and absence of the tight junction modulating agent, Sulfobetaine 16. Animal subjects were administered either a single dose of SAMe disulfate tosylate in combination with inactive excipients and Sulfobetaine 16 formulated at a 1:1 ratio (w/w) in an enteric-coated capsule, or the control consisting of the identical formulation but without Sulfobetaine 16; and

FIG. 4B is a graph showing the average plasma concentration of SAMe versus time when SAMe is delivered both in the presence and absence of the tight junction modulating agent, Sulfobetaine 14. Animal subjects were administered either a single dose of SAMe disulfate tosylate in combination with inactive excipients and Sulfobetaine 14 formulated at a 1:1 ratio (w/w) in an enteric-coated capsule, or the control consisting of the identical formulation but without Sulfobetaine 14.

FIG. 5 is a graph of the average plasma concentration of SAMe as well as the SAMe metabolite, S-adenosyl homocysteine (SAH), versus time from seven subjects administered a 1600 mg dose of commercially available SAMe tosylate disulfate;

FIG. 6 is a graph of the average plasma concentration of SAMe versus time from seven subjects each administered a 400 mg dose of an uncoated oral formulation of SAMe.

### DETAILED DESCRIPTION OF THE INVENTION

The inventors have discovered that contrary to the general state of the art, SAMe bioavailability is low because it is poorly absorbed and also that SAMe demonstrates distinct absorption patterns within different regions of the GI tract in humans. Furthermore, they found that the level of SAMe metabolites in the blood are minimally affected upon exogenous SAMe administration, which also clearly suggests that low bioavailability of exogenous SAMe is not primarily due to extensive first pass metabolism in the liver. Finally, known tight junction modulators significantly increase the permeability of SAMe across a model monolayer of cells and in vivo delivery of certain tight junction opening absorption enhancers in combination with exogenous SAMe result in substantial increases in SAMe bioavailability. The importance of these discoveries is significant and several techniques are provided herein which can alter and increase the gastrointestinal absorption of SAMe.

The inventors discovered that because SAMe permeability is low, it is possible to increase SAMe bioavailability in vivo by utilizing factors which enhance the absorption rate of this compound.

Some exemplary embodiments relate to compositions that modulate and improve the absorption and bioavailability of non-parenterally administered SAMe. Related exemplary embodiments provide methods of using the compositions for therapeutic treatment of certain diseases and/or disorders and/or as nutritional supplements and/or as medical foods. Additional embodiments relate to combinations of SAMe with one or more active ingredients that are commonly prescribed or used for treatment of and/or prophylaxis of various diseases or disorders in a subject.

As used herein the term “SAMe” refers to S-adenosyl-L-methionine and its variant, S-adenosylmethionine. As shown in the structural formula presented earlier, SAMe appears as a charged species, and its ionization state varies with pH. As mentioned previously, in its solid form, SAMe is present as a salt comprised of the SAMe ion as well as one or more counter-ions. It is common to find SAMe in a stable salt form (e.g. with p-toluenesulfonic acid as the negative counter ion) alone or in combination with one or more additional salt-forming substances, for example, mineral or organic acids and/or amino acids (See U.S. Pat. No. 3,893,999, incorporated herein by reference in its entirety). Other stable SAMe salts are described in, for example, U.S. Pat. No. 5,128,249, which discloses particular stable salts of SAMe. Various morphologies of SAMe are suitable for use in the present embodiments. Thus, as used herein “SAMe” refers to the stable salts and amorphous forms and semicrystalline forms and crystalline forms of SAMe as well as to the ionic form of SAMe when present in vivo. Amorphous forms of SAMe can be employed at any particle size and particle size distribution.

In certain embodiments, formulations for non-parenteral administration of SAMe are provided as solid or semi-solid products or dosage forms, such as tablets, capsules or pellets, and generally consist of a core “matrix material” which ‘encapsulates’ the drug as well as one or more protective coatings. “Product” or “dosage form” as used herein refers to any solid or semi-solid formulation or preparation used for non-parental administration of SAMe. In certain embodiments, non-parenteral formulations or preparations as described herein include oral delivery systems exemplified by tablets, pastes, capsules, granules, caplets, lozenges and the like; and transdermal, transmucosal or inhaled delivery systems, exemplified by aerosols, irrigants, topical creams, pastes, patches, lozenges and the like, all of which are well-known and well-documented in the art. In some embodiments, these formulations may be administered using a clinical, pharmaceutical or veterinary dosing regimen. In some embodiments, non-parenteral SAMe dosage forms may also be provided as medical foods or dietary or nutritional supplements.

In certain embodiments, non-parenterally administered SAMe formulations may be configured to enable extended release of the encapsulated SAMe. Co-owned U.S. patent application 2009/0088404, which is incorporated herein by reference, provides novel formulations of extended-release SAMe formulations. As disclosed in U.S. 2009/0088404, there are a variety of methods which can be used to prepare extended-release compositions of various types of drugs; and it is contemplated that at least one of these methodologies can be used to prepare extended-release SAMe compositions with enhanced bioavailability properties. The types of extended-release SAMe compositions that are contemplated within the scope of the present embodiments include osmotic dosage forms, extended-release matrices, pulsatile-release formulations and extended-release formulations coated with one or more enteric coatings all of which are described in detail in U.S. 2009/0088404.

A “physiologically effective dosage” of SAMe as used herein is meant to include an amount of SAMe which is administered under a defined dosing regimen for either clinical, pharmaceutical, medicinal, veterinary, dietary or nutritional purposes. Thus a “physiologically effective dosage” of SAMe includes a therapeutically effective dosage, a pharmaceutically acceptable dosage, a veterinary acceptable dosage, a nutraceutically acceptable dosage, a dietary acceptable dosage and a nutritionally acceptable dosage of SAMe as well as an acceptable dosage for use as a medical food and all of which are included for use in the present embodiments.

In certain embodiments, the relative bioavailability of SAMe formulations is determined by assessing its pharmacokinetic profile using well known techniques such as area under the curve (AUC; which is a measure of the overall exposure of a subject to SAMe in the plasma after a dose), Cmax (i.e. the highest concentration of SAMe in the plasma that is measured after a dose and Tₘₐₓ (i.e. the time after administration of a drug when the maximum plasma SAMe concentration is reached)—all of these measurements are extensively described in the art.

In some embodiments, provided herein is a method for treating and/or prophylaxis in a subject a disorder selected from the group consisting of, but not limited to, a mental or psychiatric disorder (e.g. psychotic/mood or non-psychotic mental disorders exemplified by depression and substance related disorders, respectively), a nervous system disease/disorder (e.g. a central nervous system disease exemplified by Alzheimer's), other neurological disease/disorders (e.g. headaches and sleep disorders), conditions associated with injury to the central nervous system, a liver disease/disorder (e.g. alcoholic liver disease), a cancer (e.g. solid and blood-borne cancers), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper- or hypo-methylation, a gastrointestinal disease/disorder, a cardiovascular disease/disorder, a metabolic disease/disorder (e.g. Type 2 diabetes) and a disorder induced in whole or in part by oxidative or free-radical damage, comprising administering to said subject an exemplary composition provided herein which enhances the absorption of a physiologically effective dosage of SAMe, whereby the enhanced-absorption provides an increase in SAMe bioavailability.

Some exemplary embodiments relate to compositions and methods of their use for enhancing the effectiveness of a physiologically effective dosage of SAMe utilized as a dietary or nutritional supplement in a subject. In some embodiments, effectiveness as a dietary or nutritional supplement may be measured using one or more nutritional performance variables, such as improved concentration, memory, mood, nutritional status or liver status.

### Absorption-Enhancing Technologies as a Means of Improving SAMe Absorption and Bioavailability

Through the discovery that SAMe absorption is a limiting factor in the systemic bioavailability of SAMe, provided herein are means of increasing or modulating its absorption. Any method which either directly or indirectly enhances SAMe absorption throughout the body is contemplated within the scope of the present embodiments, including for example, increasing SAMe residence time in the GI tract thereby allowing more opportunity for uptake, delivering SAMe to targeted regions of the GI tract that exhibit increased drug absorption characteristics, incorporation of “absorption enhancers” (including “penetration enhancers” and “promoters”) which increase either transcellular and/or paracellular transport of drugs (including agents which directly affect tight junctions); encapsulating SAMe in nanocarriers that deliver SAMe directly to cells; maintaining SAMe in its cationic form, modulating diet and/or dosing schedule, delivering SAMe uncoated or a combination of any of such ‘technologies’ which modulate absorption. When referring to the “gastrointestinal tract” or “GI tract”, it is intended to include the entire region beginning with the mouth/cheeks through to the esophagus, stomach, small intestine, large intestine and colorectal regions.

### Mechanisms to Increase Gastric Retention Time

In certain embodiments, increasing SAMe gastric retention time may be achieved using, for example, gastroretentive dosage forms (GRDF) of the drug including floating, geometric, bioadhesive and swelling dosage forms which are designed to withstand peristalsis and mechanical contractility of the stomach.

### GI Segment-Specific Targeted Formulations

In certain embodiments, site-specific delivery of SAMe to multiple sites along the GI tract is useful in understanding and modulating SAMe absorption since the unique environment of different segments throughout the intestinal tract can affect absorption of different drugs. In particular, drugs which show low permeability in the GI tract tend to be absorbed in specific areas along the tract. Thus, in some embodiments, their delivery site may be controlled in order to control the absorption.

In certain embodiments, targeted delivery sites in the GI tract include one or more of the mouth, stomach, duodenum, jejunum, ileum, colon and rectum. The pH along the GI tract varies from as low as 1 in the stomach to 8 in certain segments of the intestines. The GI tract is a highly complex environment with distinct pH zones that vary in location depending on a number of factors, including diet. Typically the pH ranges from lowest in the stomach to higher pH zones in the small and large intestine.

The large intestine is the final organ comprising the GI tract and includes the colon and rectum. The large intestine is the site for water resorption and formation of feces. Like the buccal area, blood that drains the rectum is not first transported to the liver. Therefore, absorption that takes place in the rectum (e.g., from rectal suppositories and enemas) enters the systemic circulation system without any biotransformation that may otherwise have occurred in the liver.

In addition to pH and/or tight junction porosity, other physiological factors such as surface area, enzymatic and transporter activity, as well as colonic microflora influence drug absorption, and it is within the scope of the present embodiments to modulate one or more of these factors in any region(s) of the GI tract as a means of affecting the bioavailability of SAMe.

Paracellular transport, mediated through tight junctions is higher in the proximal segments of the GI tract, exemplified by the duodenum, jejunum and ileum. Paracellular transport is much less in the distal segments, such as the colon.

Some exemplary embodiments relate to novel compositions comprising pH-dependent coated SAMe in combination with at least one absorption enhancing agent, wherein the composition of the pH-dependent coating acts to release a physiologically acceptable dosage of SAMe in segment-specific areas of the GI tract. In some embodiments, pH-dependent coatings may be configured to enable release of SAMe in several regions along the entire GI tract in order to affect site-specific absorption and bioavailability of SAMe.

Some exemplary embodiments relate to compositions comprising SAMe in non-enteric coated (or “uncoated”) formulations. In contrast to the current general state of the art, the inventors found that SAMe can be effectively released into the stomach and give rise to modestly elevated SAMe plasma levels and therefore an enteric coating is not critical for preventing degradation.

### Absorption Enhancers

The epithelial and endothelial barriers of the human body provide major obstacles for drug delivery to the systemic circulation systems and also to organs with unique environments, such as the central nervous system. Several transport routes exist in these barriers, which potentially can be exploited for enhancing drug permeability and absorption. Compared to the transcellular pathways (via transporters, adsorptive and receptor-mediated transcytosis), the paracellular flux for cells and molecules is very limited. Over the past 40 years many groups have been developing absorption or permeability enhancers. These “promoters” are generated as a means of modifying intercellular junctions and paracellular permeability.

Thus, some exemplary embodiments relate to compositions comprising a physiologically acceptable dosage of SAMe in combination with one or more “absorption enhancers”. “Absorption enhancers,” such as paracellular permeability enhancers (PPE) or “promoters” typically fall into the broad chemical categories of detergents or surfactants (such as zwitterionic surfactants), non-surfactants (such as unsaturated cyclic ureas), fatty acids, bile acids and chelating agents. Each agent may improve absorption of orally delivered active ingredients, by one or more mechanisms exemplified by altering the rheology of the overlying mucous, fluidizing the cell membrane lipid bilayer, affecting the tight junctional complex, inhibiting enzyme or transporter activity, influencing the drug itself in some way, among others. In certain embodiments, absorption enhancers used herein may function through a number of chemical or physical interactions including those that: (1) modulate SAMe solubility; (2) improve SAMe mucous diffusivity; (3) protect SAMe from pH, lumenal and/or brush border enzymes; (4) protect SAMe from nonspecific binding sites; and (5) improve SAMe's permeability through the mouth and/or gastrointestinal epithelium.

Examples of absorption enhancers which are suitable for use in the present embodiments include, but are not limited to, small molecule enhancers that are commonly referred to as CPEs (chemical penetration enhancers; as listed in Table 1 below), bile salts, surfactants, phospholipids, glycerides and fatty acids, as well as peptide hormones, cytoskeletal perturbing agents, oxidants, calcium ion (Ca⁺⁺) chelators and ionophores.

TABLE 1
List of CPEs

| | | | CAS |
|---|---|---|---|
| Abbreviations | Chemical Name | Category | number |
| SLS | Sodium lauryl sulfate | AS | 151-21-3 |
| SDS | Sodium decyl sulfate | AS | 142-87-0 |
| SOS | Sodium octyl sulfate | AS | 142-31-4 |
| SLA | Sodium laureth sulfate | AS | 68585-34-2 |
| NLS | N-Lauryl sarcosinate | AS | 137-16-6 |
| CTAB | Cetyltrimethyl ammonium bromide | CS | 57-09-0 |
| DTAB | Decyltrimethyl ammonium bromide | CS | 2082-84-0 |
| BDAC | Benzyldimethyl dodecyl ammonium chloride | CS | 139-07-1 |
| TTAC | Myristyltrimethyl ammonium chloride | CS | 4574-04-3 |
| DPC | Dodecyl pyridinium chloride | CS | 104-74-05 |
| ASB | Amidosulfobetaine-16 or 3-[N,N-Dimethyl(3- | ZS | 52562-29-5 |
| | palmitoylaminopropyl) ammonio]-propanesulfonate | | |
| DPS | Decyldimethyl ammonio propane sulfonate | ZS | 15163-36-7 |
| CHAPS | 3-[(3-Cholamidopropyl)dimethylammonio]-1- | ZS | 75621-03-3 |
| | propanesulfonate | | |
| CHAPSO | 3-[(3-Cholamidopropyl)dimethylammonio]-2- | ZS | 82473-24-3 |
| | hydroxy-1-propanesulfonate | | |
| ADPS | Alkyl dimethylammoniopropane sulfonate (general | ZS | N/A |
| | class) | | |
| SB-16 | 3-(N,N-Dimethylpalmitylammonio)propanesulfonate | ZS | 2281-11-0 |
| MPS | Myristyldimethyl ammonio propane sulfonate | ZS | 14933-03-6 |
| PPS | Palmityldimethyl ammonio propane sulfonate | ZS | 2281-11-0 |
| AB | Amidobetaine (general class) | ZS | N/A |
| CAPB | Cocamidopropyl betaine or {[3- | ZS | 86438-79-1 |
| | (Dodecanoylamino)propyl](dimethyl)ammonio}acetate | | |
| | Betaine | ZS | 107-43-7 |
| DDN | N,N-Dimethyl-N-dodecylglycine betaine | ZS | 89045-97-6 |
| CBC | ChemBetaine CAS | ZS | N/A mixture |
| CAHS | Cocamidopropyl hydroxysultaine | ZS | 68139-30-0 |
| CBO | ChemBetaine Oleyl | ZS | N/A mixture |
| AO | Amine Oxide (general class) | ZS | N/A |
| LDAO | N,N-dimethyldodecylamine N-oxide | ZS | 1643-20-5 |
| PCC | Palmitoyl carnitine chloride | ZS | 6865-14-1 |
| IP | Nonylphenoxypolyoxyethylene | NS | 68412-54-4 |
| T20 | Polyoxyethylene sorbitan monolaurate | NS | 9005-64-5 |
| T40 | Polyoxyethylene sorbitan monopalmitate | NS | 9005-66-7 |
| SP80 | Sorbitan monooleate | NS | 1338-43-8 |
| TX100 | Triton-X-100 | NS | 9002-93-1 |
| SDC | Sodium deoxycholate | BS | 302-95-4 |
| SGC | Sodium glycocholate | BS | 863-57-0 |
| CA | Cholic Acid | BS | 81-25-4 |
| HA | Hexanoic Acid | FA | 142-62-1 |
| HPA | Heptanoic Acid | FA | 111-14-8 |
| LME | Methyl Laurate | FE | 111-82-0 |
| IPM | Isopropyl myristate | FE | 110-27-0 |
| IPP | Isopropyl palmitate | FE | 142-91-6 |
| MPT | Methyl palmitate | FE | 112-39-0 |
| SDE | Dibutyl sebacate | FE | 109-43-3 |
| SOA | Sodium oleate | SS | 143-19-1 |
| UR | Urea | FM | 57-13-6 |
| LAM | Lauryl amine | FM | 124-22-1 |
| CL | Caprolactam | NR | 105-60-2 |
| MP | Methyl pyrrolidone | NR | 872-50-4 |
| OP | Octyl pyrrolidone | NR | 2687-94-7 |
| MPZ | Methyl piperazine | NR | 109-01-3 |
| PPZ | Phenyl piperazine | NR | 92-54-6 |
| EDTA | Ethylenediaminetetraacetic acid | OT | 60-00-4 |
| SS | Sodium salicylate | OT | 54-21-7 |
| CP | Carbopol 934P | OT | 9003-04-7 |
| GA | Glycyrrhetinic acid | OT | 471-53-4 |
| BL | Bromelain | OT | 9001-00-7 |
| PO | Pinene oxide | OT | 1686-14-2 |
| LM | Limonene | OT | 5989-27-5 |
| CN | Cineole | OT | 470-82-6 |
| ODD | Octyl dodecanol | OT | 5333-42-6 |
| FCH | Fenchone | OT | 7787-20-4 |
| MTH | Menthone | OT | 14073-97-3 |
| TPMB | Trimethoxy propylene methyl benzene | OT | 2883-98-9 |

AS Anionic surfactants,
CS cationic surfactants,
ZS zwitterionic surfactants,
NS nonionic surfactants,
BS bile salts,
FA fatty acids,
FE fatty esters,
FM fatty amines,
SS sodium salts of fatty acids,
NR nitrogen-containing rings,
OT others.

Recent advances in drug absorption research led to the discovery of an increasing number of integral membrane, adaptor, regulator and signaling proteins in tight and adherens junctions. Tight junctions are intercellular junctions between cells that form a barrier between the cells. In this way, materials (e.g. small molecules, proteins and drugs) cannot pass between cells but rather must be taken up by the cell and thus enables the cells to regulate what is allowed through. Tight junctions occur in many regions throughout the body including the mouth, small intestine, large intestine and colon and vary in density/tightness within different regions. Within the GI tract, tight junctions refer to the areas between adjacent endothelial cells and act, in part, to regulate the uptake of digested materials. Tight junctions are highly regulated and are one of the key elements that form the barrier between the luminal environment of the mouth and/or GI tract and the rest of the body.

Tight junctions have three main functions: (1) to hold cells together, (2) to block the movement of integral membrane proteins between the apical and basolateral surfaces of the cell, allowing the specialized functions of each surface (for example receptor-mediated endocytosis at the apical surface and exocytosis at the basolateral surface) to be preserved (this aims to preserve the transcellular transport) and (3) to prevent the passage of molecules and ions through the space between cells and therefore materials must actually enter the cells (by diffusion or active transport) in order to pass through the tissue. This pathway provides control over what substances are allowed through.

New tight junction modulators or opening agents are currently under development, which can directly target tight or adherens junction proteins, the signaling pathways regulating junctional function, or tight junction associated lipid raft microdomains. Modulators acting directly on tight junctions include peptides derived from zonula occludens toxin, Clostridium perfringens enterotoxin, peptides selected by phage display that bind to integral membrane tight junction proteins, and lipid modulators. They can reversibly increase paracellular transport and drug delivery and have a potential to be used as pharmaceutical excipients to improve drug delivery across epithelial barriers and the blood-brain barrier. Exemplary “tight junction modulators” suitable for use in the present embodiments include, but are not limited to, chitosan, poly(acrylic acid), cytochalasin D; caprate, spermine, taurocholate (including sodium and other salt forms) and other bile acids and/or their salts (such as cholic acid, sodium cholate or potassium cholate), as well as more recently identified agents which include peptides derived from zonula occludens toxin or Clostridium perfringens enterotoxin. Classes of tight junction modulators included herein thus include: saturated and/or unsaturated fatty acids or their corresponding carboxylate salts (e.g. C6-C24 fatty acids, or carboxylate salts thereof, especially C8-C22 fatty acids, or carboxylate salts thereof, C10-C20 fatty acids or carboxylate salts thereof, C6-, C7-, C8-, C9-, C10-, C11-, C12-, C13-, C14-, C15-, C16-, C17-, C18-, C19-, C20-, C21-, C22-fatty acids or carboxylate salts thereof), saturated and unsaturated sulfonic acids and sulfonate salts thereof (e.g. C6-C24 sulfonic acids or sulfonate salts, especially C8-C22 sulfonic acids or sulfonate salts, C10-C20 sulfonic acids or sulfonate salts, C8-, C9-, C10-, C11-, C12-, C13-, C14-, C15-, C16-, C17-, C18-, C19-, C20-, C21-, C22-sulfonic acids or sulfonate salts); zwitterionic surfactants (e.g. 3-(N,N-Dimethylpalmitylammonio)propanesulfonate, decyldimethyl ammonio propane sulfonate, myistyldimethyl ammonio propoane sulfonate, cocamidopropyl hydroxysultaine (ChemBetaine® CAS), oleyl betaine (ChemBetaine®Oleyl), or palmitoyl carnitine chloride); fatty amines (e.g. C6-C24 fatty amines, especially C8-C22 fatty amines, C10-C20 fatty amines, C6-, C7-, C8-, C9-, C10-, C11-, C12-, C13-, C14-, C15-, C16-, C17-, C18-, C19-, C20-, C21-, C22-fatty amines), as well as other organic acids (e.g. tartaric acid) and cyclodextrins (e.g. alpha-cyclodextrin, beta-cyclodextrin, or gamma-cyclodextrin). Exemplary fatty acids that may be used include hexanoic, heptanoic, capric, lauric acid, myristic acid, palmitic acid, stearic acid, arachidic acid, myristoleic acid, palmitoleic acid, sapienic acid, oleic acid, linoleic acid, α-linolenic acid, arachidonic acid, eicosapentaenoic acid, erucic acid, docosahexaenoic acid. Exemplary carboxylate salts that may be used include sodium or potassium caprate, caprylate, laurate, myristate, palmitate, stearate, arachidate, myristoleate, palmitoleate, sapienate, oleate, linoleate, α-linolenate, arachidonate, eicosapentaenoate, erucate, docosahexaenoate. Specific carboxylate salts include sodium caprate, sodium caprylate, and sodium laurate. Specific fatty amines that may be used include lauryl amine (N-dodecylamine), decylamine, nonylamine, octylamine, heptylamine or hexylamine. Exemplary sulfonic acids that may be used include octane sulfonic acid, decane sulfonic acid (e.g. sodium 1-decanesulfonate), dodecane sulfonic acid, tetradecane sulfonic acid, hexadecane sulfonic acid, octadecane sulfonic acid, eicosane sulfonic acid, docosane sulfonic acid or tetracosane sulfonic acid. Specific sulfonic acids that may be mentioned include dioctyl sodium sulfosuccinate.

Zwitterionic agents, for example zwitterionic surfactants as listed above, are preferred absorption enhancing agents in some embodiments. These agents are compounds with a net neutral charge, which have at least one positive and one negative electrical charge on different atoms within the molecule. They are sometimes referred to as inner salts or dipolar ions. Zwitterionic agents for use in certain embodiments are generally neutral at physiologically relevant pH levels (i.e. about pH 6-8). A preferred zwitterionic surfactant for use in the certain embodiments is an acyl carnitine, more preferably, a long chain acyl carnitine, and even more preferably, palmitoyl carnitine. Some of these agents are naturally occurring molecules that are intermediate structures, formed by the body to assist in the intracellular transport of long chain acyl groups to the mitochondria for energy production, though synthetic analogs may also be prepared. See the formula below for the general structure as well as other examples of long chain acyl carnitines which may be used in the present embodiments:

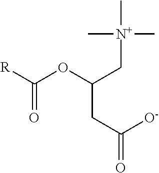

| Name | R Structure | R = |
|---|---|---|
| Stearoyl carnitine | 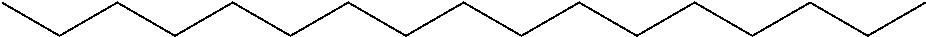 [SMILES: CCCCCCCCCCCCCCCCC] | C₁₇H₃₅ |
| Palmitoyl carnitine | 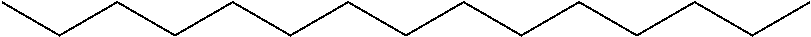 | C₁₅H₃₁ |
| Myristoyl carnitine | 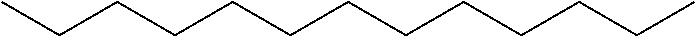 | C₁₃H₂₇ |
| Lauroyl carnitine | 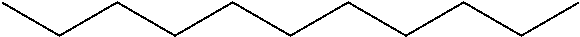 | C₁₁H₂₃ |
| Decanoyl carnitine | 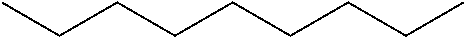 | C₉H₁₉ |

Thus, some embodiments comprise a combination of SAMe and one or more acyl carnitines of the above formula in which the R is CH₃(CH₂)ₙ, n is 7-21, preferably 9-17, and particularly 7, 8, 9, 10, 11, 12, 13, 14, 15, 16, 17, 18, 19, 20 or 21.

Certain embodiments thus relate to examining the effect of acyl carnitine chain length in in vitro cell permeability of SAMe in appropriate models (e.g. Caco-2) and also the effect of chain length on the pharmacokinetic properties in animal models (e.g. Beagle dogs) in solid or semi-solid oral dosage forms.

Another example of zwitterionic surfactants included for use in the present embodiments are the long alkyl chain N substituted dimethylammoniopropane sulfonates (N substituted-N,N-dimethyl-3-ammonio-1-propane sulfonates). These molecules are sometimes referred to as the sulfobetaines. See the formula below for the general structure as well as other examples of long chain dimethylammoniopropane sulfonates which may be used in the present embodiments:

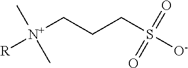

| Name | R Structure | R = |
|---|---|---|
| Sulfobetaine 18 | 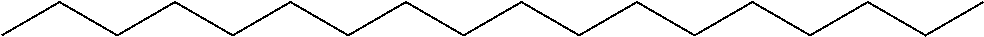 [SMILES: CCCCCCCCCCCCCCCCCC] | C [symbol] H [symbol] |
| Sulfobetaine 16 | 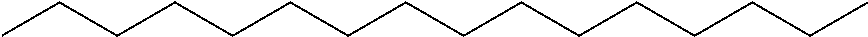 [SMILES: CCCCCCCCCCCCCCCC] | C [symbol] H [symbol] |
| Sulfobetaine 14 | 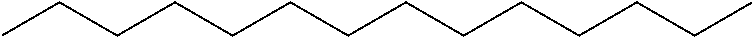 | C [symbol] H [symbol] |
| Sulfobetaine 12 | 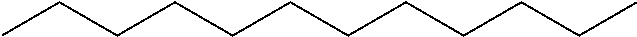 | C [symbol] H [symbol] |
| Sulfobetaine 10 | 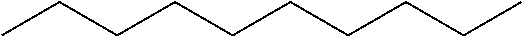 | C [symbol] H [symbol] |

[symbol]

indicates data missing or illegible when filed

Thus, some exemplary embodiments comprise a combination of SAMe and one or more dimethylammoniopropane sulfonates of the above formula in which the R is CH₃(CH₂)ₙ, n is 7-21, preferably 9-19, and particularly 7, 8, 9, 10, 11, 12, 13, 14, 15, 16, 17, 18, 19, 20 or 21.

Other examples of zwitterionic surfactants included for use in the present embodiments are listed with their general structures in the table below:

| zwitterionic/amphoteric surfactant | General Structure |
|---|---|
| alkyl betaines | 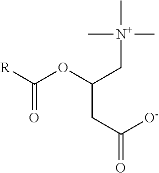 |
| Alkyl lamidobetaines | 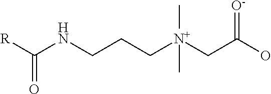 |
| Alkyl amine oxides | 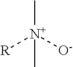 |
| 3-[N,N-Dimethyl(3-acylaminopropyl) ammonio]-propanesulfonates | 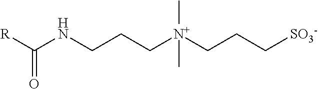 |
| alkylamidopropylhydroxysultaines | 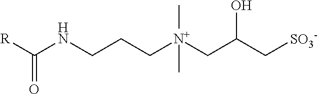 |

Thus, some embodiments comprise a combination of SAMe and one or more of these zwitterionic surfactants of the formulas shown in the table above, in which the R is CH₃(CH₂)ₙ, n is 7-21, preferably 9-19, and particularly 7, 8, 9, 10, 11, 12, 13, 14, 15, 16, 17, 18, 19, 20 or 21.

The inventors show for the first time that a subset of high potency tight junction opening absorption enhancers, which increase SAMe absorption when used at very low levels, exists. Furthermore, identification of potential high potency tight junction opening agents may be carried out using, for example, a SAMe Caco-2 cell model as described in the Examples. Based on the concentration of tight junction modulator utilized as well as the increase in SAMe permeability achieved, suitable high potency tight junction modulators elicit at least a two-fold increase in SAMe uptake in vitro when used at low concentrations (i.e. about 2 mM or below). More preferably, at least a three-fold increase in SAMe uptake occurs at low concentrations of these tight junction opening agents and, even more preferably, a four-fold or greater increase is achieved.

Other zwitterionic surfactants for use in the present embodiments also have long alkyl chains as a structural feature and particular groups of molecular analogs, differing only in alkyl chain length, will be investigated.

Thus certain exemplary embodiments relate to compositions comprising a physiologically effective dosage of SAMe and at least one zwitterionic surfactant. In some exemplary embodiments said zwitterionic surfactant is an acyl carnitine. In other exemplary embodiments said zwitterionic surfactant is an N substituted dimethylammoniopropane sulfonate or sulfobetaine. In preferred embodiments, said zwitterionic surfactant is co-formulated with the physiologically acceptable dosage of SAMe. Other exemplified embodiments relate to compositions comprising a physiologically effective dosage of SAMe and at least one high potency tight junction opening enhancer.

In some embodiments, the solid oral composition contains a physiologically effective dose of S-adenosylmethionine (SAMe) and a tight junction opening enhancer, which enhances delivery of SAMe, as measured by an increase in SAMe Cmax and/or AUC. The increase in SAMe Cmax may be determined by comparison of SAMe delivery of the composition comprising SAMe and the absorption enhancer (test dosage) with delivery of a SAMe using a comparable control, which contains the same amount of SAMe but lacks the absorption enhancer. In some embodiments, the composition comprising SAMe and the absorption enhancer is substantially similar to the control, with the exception that the control lacks the absorption enhancer. (In this context, “substantially similar” means that the test dosage and the control contain the same amount of SAMe in the same dosage form with the same types of excipients.) Likewise, the increase in AUC may be determined by comparing SAMe delivery using the composition comprising SAMe and the absorption enhancer with delivery of a SAMe using the control. In some embodiments, the SAMe Cmax obtained with the composition comprising SAMe and enhancer may be at least 110%, at least 120%, at least 130%, at least 140%, at least 150%, at least 160%, at least 180%, at least 200%, at least 300%, at least 400% or at least 500% of the SAMe Cmax obtained with the control. Likewise, in some embodiments the SAMe AUC obtained with the composition comprising SAMe and enhancer may be at least 110%, at least 120%, at least 130%, at least 140%, at least 150%, at least 160%, at least 180%, at least 200%, at least 300%, at least 400% or at least 500% of the SAMe AUC obtained with the control. In certain embodiments, the SAMe Cmax and/or AUC in the presence of a tight junction opening enhancer is at least 10%, at least 20%, at least 30%, at least 40%, at least 50%, at least 60%, at least 70%, at least 75%, at least 80%, at least 85%, at least 90%, at least 95%, at least 100%, at least 150%, at least 200%, at least 300%, at least 400%, or at least 500% higher than the Cmax of the control group. In certain embodiments, the SAMe Cmax and/or AUC in the presence of a tight junction opening enhancer is at least 2-fold, at least 3-fold, at least 4-fold, at least 5-fold, at least 10-fold higher than the Cmax of the control group. In some embodiments, the Cmax and/or AUC are determined in a suitable test population comprising at least six subjects without outliers. In some embodiments, the suitable test population is a population of mammals, such as humans, dogs, cats, rats, mice, monkeys or other mammalian test subjects. In some embodiments, the test subjects are humans. In some embodiments, the test subjects are dogs.

Pharmacokinetic parameters such as average maximum plasma concentration of SAMe (Cmax) are determined using a bioanalytical method with adequate sensitivity, specificity, ruggedness, stability and repeatability (for example, a qualified liquid chromatography triple quad mass spectrometry based method coupled with a suitable extraction method for the separation of analyte from plasma). SAMe AUC values were calculated from 0-24 hours using the trapezoid method and are uncorrected for baseline, endogenous SAMe levels. A “suitable test population” preferably has six or more subjects who are dosed fasted. All members of the “suitable test population” have pharmacokinetic parameters for SAMe that fall within statistically normal ranges (i.e. no outliers) and no member will be included on the basis of non-standard or unusual SAMe absorption or metabolism. The average Cmax values are preferably derived by averaging the individual Cmax concentrations for each member of the subject group. Use of present compositions and methods in vivo provides high SAMe Cmax and/or AUC values in comparison to conventional dosage forms of SAMe. For greater clarity all references to dose within this patent refer to dose as the dose of SAMe ion.

Tight junction modulators may be particularly effective in improving modified release dosage forms targeting more distal segments of the GI tract. The porosity of tight junctions is tighter in distal segments such as the ileum and colon, compared to, for example, the duodenum (i.e. tight junctions of the duodenum are more porous than those of the lower GI segments.) In addition, transit time in the upper GI tract is faster than in the lower GI tract. The combinations of less porous tight junctions coupled with the slower transit time in the lower segments suggest that the use of tight junction modulators in the lower GI tract may be more impactful on a relative basis. This effect could be expected to extend SAMe delivery (as well as increase its absorption using tight junction modulators) in the colon and in the case of suppository formulation, to the rectum, which would constitute targeted delivery to a desired site of action in the treatment of colon and/or other gastric cancers. This distally targeted delivery is optimal to exploit the antineoplastic properties of SAMe on colon and/or other gastric cancer cells. Thus in some embodiments, compositions comprising a delayed release component, such that the release of SAMe is configured to dissolve in the large intestine or colon, are included. Other embodiments include methods of treating a condition related to the colon or large intestine, which condition is amenable to treatment with SAMe, comprising administering to a patient a physiologically effective amount of a SAMe and at least one absorption enhancing agent. Preferably, said absorption enhancing agent is a tight junction modulating agent.

Improved buccal delivery of SAMe is also considered practical using formulations provided herein comprising one or more tight junction modulators considering the presence of tight junctions in the mouth. Thus non-parenteral formulations provided herein are meant to include those which target buccal, upper and lower intestinal regions including the colon and rectum.

Thus in certain embodiments, provided herein are compositions for buccal delivery comprising a physiologically acceptable dosage of SAMe and at least one tight junction modulator.

In some embodiments the composition is administered as a buccal dosage form. In other embodiments the composition is administered as a suppository.

In certain embodiments, provided herein is a lower GI targeted modulator-enhanced component in combination with an upper GI-targeted conventional or modulator-enhanced component to create a modified release dosage form with enhanced absorption over an extended period of time.

Preferably, the suitability of a particular “absorption enhancer”, including “tight junction modulators,” will be identified in vitro by use of SAMe cellular permeability studies. Most relevant cell lines will suffice for such in vitro experimentation including, but not limited to, Caco-2 cells (as described in Example 3). In addition, the use of references in the art may also provide insight into potentially suitable “absorption enhancers” or “tight junction modulators” for use in the present embodiments.

### Nanocarriers to Increase Delivery of SAMe

In certain embodiments, encapsulating SAMe into nano-sized carriers which are suitable for use in non-parenteral administration of SAMe (e.g. nanoparticles and colloidal systems) may result in increased delivery to the cells. Several approaches have been described that appear to increase transcellular intestinal absorption without damaging the epithelium. These approaches can be categorized into methods that stabilize the drug, increase drug solubility or alter its characteristics to improve transcellular permeability. Various colloidal systems which may be suitable for enhancing the absorption of SAMe are exemplified by sub-micron emulsions, polymeric nanoparticles, microparticles, and the like. There are various physicochemical factors governing gastrointestinal uptake of such systems including size, size distribution, consistency, hydrophobicity and surface properties which may be modulated in order to enhance SAMe cellular uptake.

Dosing with Formulations Exhibiting Enhanced-Absorption and Bioavailability of SAMe

In some embodiments the enhanced-absorption SAMe formulations provided herein relate to enhanced nutritional support, or dietary supplement health improvements including, but not limited to, mood improvement, joint health and liver function. In some exemplary embodiments the disorder is related to the dietary management of a disease through additional supplementation of SAMe which cannot be reached through diet (e.g. a “medical food”.)

Some exemplary embodiments relate to a method for treating and/or prophylaxis in a subject a disease or disorder selected from the group consisting of, but not limited to, a mental or psychiatric disorder (e.g. psychotic/mood or non-psychotic mental disorders exemplified by depression and substance related disorders, respectively), a nervous system disease/disorder (e.g. a central nervous system disease exemplified by Alzheimer's, Progressive Supranuclear Palsy or other Tauopathy disorders), other neurological disease/disorders (e.g. headaches and sleep disorders), conditions associated with injury to the central nervous system, a liver disease/disorder (e.g. alcoholic liver disease), a cancer (e.g. solid and blood-borne cancers), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper- or hypo-methylation, a gastrointestinal disease/disorder, a cardiovascular disease/disorder, and a disorder induced in whole or in part by oxidative or free-radical damage, comprising administering to said subject an exemplary composition which enhances the absorption and bioavailability of a physiologically effective amount of exogenous SAMe.

Some embodiments relate to therapeutic use of the exemplary compositions disclosed herein for treatment of a mental or psychiatric disorder selected from the group consisting of anxiety disorders, depressive disorders, eating disorders, bipolar disorder, abuse disorders, dependence disorders, Axis II disorders, and psychosis. In some exemplary embodiments, the mental or psychiatric disorder is an anxiety disorder selected from the group consisting of generalized anxiety disorder, posttraumatic stress disorder, social anxiety disorder, panic disorder, Schizophrenia and obsessive compulsive disorder. In some exemplary embodiments, the mental or psychiatric disorder is a depressive disorder selected from the group consisting of major depressive disorder, multi-infarct dementia, minor depression, postpartum or late-life depression (and the like), Parkinson's depression, HIV-associated depression, brief recurrent depression, dysthymia or depression NOS (Not Otherwise Specified). In some exemplary embodiments, the mental or psychiatric disorder is an eating disorder selected from the group consisting of bulimia nervosa, anorexia nervosa, binge eating disorder, obesity, or eating disorder NOS. In some exemplary embodiments, the mental or psychiatric disorder is bipolar disorder, an abuse disorder or a dependence disorder, including abuse of, or dependence on, alcohol, nicotine, cocaine, codeine, oxycodone, hydrocodone or other opiates. In some exemplary embodiments, the mental or psychiatric disorder is an Axis II disorder selected from borderline personality disorder.

In some exemplary embodiments, the disorder is a nervous system disorder, including a central nervous system (CNS) disorder such as Parkinson's disease, Alzheimer's disease, Tauopathy disorders (such as, for example, Progressive Supranuclear Palsy (PSP)), Angelman Syndrome (genetic disorder), Multiple Sclerosis (MS) and pre-dementia and/or cognitive impairment.

In some exemplary embodiments, the disorder is a comorbid disorder, such as comorbid depression arising in a subject who is undergoing treatment for one or more diseases or disorders such as but not limited to, cancer, Parkinson's, cardiovascular disease and HIV. In certain embodiments the comorbid disorder is caused by one or more therapies being utilized to treat said one or more diseases or disorders.

In some exemplary embodiments, the disorder is a drug-induced disorder or side-effect, such as anti-depressant-induced sexual dysfunction or insomnia.

In some exemplary embodiments, the disorder is a result of an injury to the CNS such as spinal cord injury or brain damage, memory loss, cognitive impairment and/or learning disability.

In some exemplary embodiments, the disorder is a liver disorder selected from the group consisting of alcoholic liver disease, fatty liver disease (non-alcoholic) hepatitis (both viral and non-viral), liver cancer, oxidative liver disease, HISS-dependent insulin resistance, cholestasis and cirrhosis.

In some exemplary embodiments, the disorder is a cancer selected from the group consisting of cancers occurring in one or more of the liver, colon, rectum, ovaries, urethra, testicles, bladder, breast, stomach, esophagus, pancreas, head and neck, lung, blood, skin (such as actinic keratosis, basal cell cancer, superficial basal cell cancer, squamous cell cancer, and melanoma) and adenocarcinomas.

In some exemplary embodiments, the disorder is a joint disorder such as, for example, arthritis and osteoarthritis.

In some exemplary embodiments, the disorder is an inflammatory disorder selected from the group comprising systemic lupus erythematosis, Reye's syndrome, rheumatic fever, allergic rhinitis, myasthenia gravis, temporal arteritis, vasculitis, psoriasis, atopic dermatitis, rosacea, eczema, alopecia universalis, scleroderma, pemphigus, contact dermatitis, ankylosing spondylitis, dermatomyositis, polymyositis, celiac sprue, Guillain-Barré syndrome, multi-infarct dementia, post-cerebral vascular accident reperfusion damage, Addison's disease, Hashimoto's thyroiditis, asthma, upper respiratory inflammation symptoms, chronic bronchitis, atherosclerosis, pernicious anemia, autoimmune hepatitis, prostatitis, pelvic inflammatory disease, Goodpasture's syndrome, Wegener's granulomatosis, chronic nephritis, Sjogrens syndrome, or allergic conjunctivitis.

In some exemplary embodiments, the disorder is a gastrointestinal disorder such as inflammatory bowel disease (IBD), Crohn's disease or ulcerative colitis (UC).

In some exemplary embodiments, the disorder is a soft tissue disease such as fibromyalgia.

In some exemplary embodiments, the disorder is a pain disorder such as fibromyalgia, chronic headaches, shingles, reflex sympathetic dystrophy and polyneuropathy.

In some exemplary embodiments, the disorder is a cardiovascular disorder which is related to hyper- or hypo-homocysteinemia such as coronary heart disease, stroke, peripheral vascular disease and atherosclerotic disease.

In some exemplary embodiments, the disorder is related to a genetic or medical condition related to a deficiency of the methylation pathway such as methylenetetrahydrofolate reductase deficiency.

In some exemplary embodiments, the disorder is a metabolic disorder, such as type 2 diabetes.

In some exemplary embodiments, the etiology of the disorder may include oxidative or free-radical damage, and is selected from the group comprising chronic fatigue syndrome, temporal arteritis, vasculitis, multi-infarct dementia, chronic emphysema, or chronic nephritis.

Among the advantages provided by enhanced-absorption SAMe formulations of the present embodiments, included are the convenience and concomitant improved subject compliance due to reduced daily dosing, an improved side-effect profile (such as decreased stomach irritation and potentially decreased tendency to induce mania in manic depressive subjects or subjects at risk for manic episodes) and other side effects associate with or caused by the relatively high doses of SAMe (typically about 400 to about 3200 mg SAMe ion/day, more typically about 800 to about 1600 mg SAMe ion/day) necessary to achieve a desired effect.

As used herein, the term “desired effect” includes a “therapeutic effect”, “pharmaceutical effect”, “dietary effect” (e.g. for use as a medical food), “nutraceutical effect” and “nutritional effect”. Thus, a “desired effect” includes ameliorating at least one symptom of a physiological disorder or disease state in a subject, or improving at least one performance variable (such as improved concentration, memory, mood, nutrition status or liver status) when used as a nutritional supplement in a subject. The “desired effect” may be achieved through nutritional supplementation using SAMe formulations provided herein or through administration using a clinical, pharmaceutical or veterinary dosing regimen of SAMe formulations provided herein.

Suitable subjects for dosing according to the methods and compositions provided herein include warm-blooded mammals such as humans, domestic or exotic animals or livestock; domesticated avian subjects such as chickens and ducks; and laboratory animals suitable for research use. When used for treating a disease or disorder in a subject, various symptoms of specific physiological disorders and disease states are contemplated as being treatable within the context of the present embodiments and details of which are set forth below. However, it is to be recognized that the understanding of various disease states by those of skill in the art is not static and this is the same for performance variables related to nutritional supplementation. Thus, though the description above is intended to be illustrative of the various disorders, disease states, symptoms or performance variables that may be treated using the enhanced-absorption SAMe formulations according to the present embodiments, a person skilled in these arts will be expected to apply such knowledge.

Dosing with Multiple Dosing Units

Some exemplary embodiments provided herein relate to treatment of and/or prophylaxis of one or more diseases in a subject, wherein the treatment of and/or prophylaxis of one or more diseases and/or disorders comprises administering to the subject an absorption-enhanced formulation comprising a physiologically acceptable dosage of S-adenosyl methionine (SAMe), or a proprietary salt thereof.

Some other exemplary embodiments provided herein relate to SAMe nutritional supplements and/or dietary supplements for improvement of one or more nutritional performance variables in a subject, wherein the nutritional performance variables are one or more of concentration, memory, mood, nutritional status and liver status, and wherein an absorption enhanced formulation comprising a physiologically acceptable dosage of 5-adenosyl methionine (SAMe), or other proprietary SAMe salts thereof, is administered to a subject.

In some exemplary embodiments, the absorption-enhanced SAMe may be divided between multiple daily doses. Multiple daily doses need not be identical and may comprise one or more dosage forms in combination. In some exemplary embodiments, the enhanced-absorption SAMe may be divided into two or more daily doses. Each dose may be administered as a single dosage unit exemplified by, a single tablet, capsule or caplet, or alternatively may be divided into multiple dosage units. In some embodiments, a twice-daily dose of from about 100 to about 1600 mg of SAMe ion per dose may be divided into one to four dosage units of from about 100 to about 800 mg of SAMe ion per unit. In each case, the form of the dosage unit may be a capsule, a tablet, a caplet (single or multi-compartment) or an extended release dosage unit and the like. In some embodiments, the absorption enhancer and SAMe are provided in a oral dosage form wherein separate compartments of the oral dosage form contain either the absorption-enhancing agent or SAMe. In other embodiments the absorption-enhancing technology is administered separately from the SAMe dosage form. Preferably, the oral dosage form is a tablet, capsule or gel-capsule.

In some embodiments, the ratio (weight:weight) of S-adenosylmethionine to tight junction opening absorption enhancer is from 1:100,000 to 100,000:1. In other embodiments, the ratio of S-adenosylmethionine to tight junction opening absorption enhancer is from 1:150 to 150:1, from 1:10 to 10:1, from 1:5 to 10:1, from 1:2 to 10:1, from 1:1 to 10:1, from 1:5 to 5:1, or from 1:2 to 2:1. In some embodiments, the ratio of S-adenosylmethionine to tight junction opening absorption enhancer is 1:1.

Conventional SAMe dosing generally administers up to 1600 mg of SAMe ion per day bi-daily (BID) in order to achieve maximum activity of the drug. Tablets are most often available commercially in 200 mg and 400 mg doses SAMe ion which require subjects to ingest 4-8 tablets per day. This is inconvenient with respect to the amount of time needed as well as the potential error in consistent dosing (i.e. if a dose is missed). Provided herein are novel compositions and methods which reduce the effective dose of SAMe (i.e. reduce the number of tablets necessary in a day to achieve the same or better efficacy as compared to conventional dosing regimens) and/or eliminate the need to dose bi-daily. By improving SAMe absorption, a new method of SAMe therapy is available which lowers the amount of SAMe dose required to elicit an effective response by providing compositions comprising one or more absorption-enhancing technologies. These exemplary “low dose” formulations may provide a lower daily pill count which is beneficial to those taking SAMe as it will reduce the time, cost and inconvenience of self-administering large doses.

Some exemplary embodiments relate to administration of the selected physiologically acceptable dosage on a once-a-day basis. In some embodiments, the once-a-day dose may be administered in a single dosage unit exemplified by, a single tablet, capsule, or caplet. In other exemplary embodiments, the single dose may be administered as multiple tablets, capsules or caplets taken at one time. In some embodiments, for instance, a dosage of about 400 to 3200 mg of SAMe per day may be divided into two, three, four or more tablets, capsules or caplets of about 50 to 2000, preferably about 100 to 1600 mg of SAMe per unit. In some preferred embodiments, the daily dose may comprise two, three or four units (e.g. tablets, capsules or caplets) of about 100 to 800 mg of SAMe ion per unit. Suitable dosage regimens included are: four units of about 50-400 mg of SAMe ion per unit, e.g. 50, 100, 150, 200, 250, 300, 350 or 400 mg SAMe ion per unit; three units of about 50-1000 mg of SAMe ion per unit, e.g. 50, 100, 150, 200, 250, 300, 350, 400, 450, 500, 550, 600, 650, 700, 750, 800, 850, 900, 950 or 1,000 mg of SAMe ion per unit; two units of about 50-1600 mg of SAMe ion per unit, e.g. about 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550 or 1600 mg of SAMe ion per unit.

Some exemplary embodiments provided herein relate to “low-dose” SAMe compositions. By increasing the bioavailability of exogenous SAMe, the daily administered dose of SAMe may be substantially lowered by administration of compositions with improved SAMe absorption. These exemplary “low-dose” treatments may enable a lower daily pill count.

Fed vs. Fasting Dose

In some embodiments, it may be advantageous to ensure that the subject is either fed or fasted (e.g. overnight for at least about 6, especially about 8, hours). It is considered that food administered at the same time, immediately (i.e. less than about 30, especially less than about 15 minutes) before or soon (e.g. less than about 10 minutes) after the absorption enhanced SAMe formulation provided herein is administered to the subject may increase or decrease the rate of gastric emptying and thus affect the rate of uptake of SAMe from the formulation. Thus, in some embodiments, provided herein are methods of administering the absorption enhanced SAMe formulation with food, wherein food is ingested either before or during SAMe treatment.

Combinations of SAMe with Other Active Ingredients

Some exemplary embodiments provided herein relate to combinations of SAMe with one or more active ingredients that can be prescribed or used for treating and/or prophylaxis in a subject a disease or disorder selected from the group consisting of, but not limited to, a mental or psychiatric disorder (e.g. psychotic or non-psychotic mental disorders such as depression and substance abuse disorders, respectively), a nervous system disease/disorder (e.g. a central nervous system disease such as Alzheimer's, Progressive Supranuclear Palsy, or other Tauopathy disorders), other neurological disease/disorders (e.g. headaches and sleep disorders), conditions associated with injury to the central nervous system, a liver disease/disorder (e.g. alcoholic liver disease), a cancer (e.g. solid and blood-borne cancers), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper or hypo methylation, a gastrointestinal disease/disorder, a cardiovascular disease/disorder, and a disorder induced in whole or in part by oxidative or free-radical damage, comprising administering to said subject an exemplary composition provided herein which enhances the absorption and bioavailability of a physiologically effective amount of exogenous SAMe.

In some exemplary embodiments provided herein relate to combinations of SAMe with one or more active ingredients that can be prescribed or used for treatment of and/or prophylaxis of mental or psychiatric disorders in a subject include, but are not limited to, tricyclic antidepressants (TCAs), tetracyclic antidepressants, aminoketones, phenylpiperazines, selective serotonin reuptake inhibitors (SSRIs), monoamine oxidase inhibitors (MAOIs), serotonin-norepinephrine reuptake inhibitors (SNRIs), norepinephrine-serotonin reuptake inhibitors (NSRIs), dopamine reuptake inhibitors, norepinephrine-dopamine reuptake inhibitors, norepinephrine reuptake inhibitors, selective serotonin reuptake enhancers, noradrenergic and serotonin specific antidepressants, substance P receptor antagonists, neurokinin receptor antagonists such as saredutant, corticotrophin release factor antagonists such as mifepristone, atypical antipsychotics such as aripiparazole, commonly used antidepressant augmenters such as lithium or triple reuptake inhibitors.

Some exemplary embodiments provided herein relate to combinations of SAMe with one or more device therapies that can be prescribed or used for treatment of and/or prophylaxis of mental or psychiatric disorders in a subject include, but not limited to ECT (electro convulsive therapy) and electric shock therapy.

Some exemplary embodiments provided herein relate to combinations of SAMe with one or more active ingredients that can be prescribed or used for treatment of and/or prophylaxis of a nervous system disease/disorder in a subject include, but are not limited to anticonvulsants such as pregabalin, α-amino-3-hydroxy-5-methyl-4-isoxazolepropionic acid (AMPA) receptor antagonists, methylphosphonate (NMPA) receptor antagonists, histamine receptor antagonists, nitric oxide (NO) modulators, glutamate receptor antagonists, acetylcholinesterase inhibitors, dopamine agonists, N-methyl-d-aspartate (NMDA) receptor antagonists such as memantine, cholinesterase inhibitors such as donepezil, neuroprotectants, nootropic agents, CNS modulators, antiamyloidogenics.

Some exemplary embodiments provided herein relate to combinations of SAMe with one or more active ingredients that can be prescribed or used for treatment of and/or prophylaxis of a liver disorder in a subject include, but are not limited to, antiviral medication such as alpha interferon, ribavirin, lamivudine, steroids, antibiotics and zinc acetate.

Some exemplary embodiments provided herein relate to combinations of SAMe with one or more active ingredients that can be prescribed or used for treatment of and/or prophylaxis of a cancer in a subject include, but are not limited to, chemotherapeutic agents, drug resistance modulators, monoclonal antibodies, cytokines (e.g. interferons and interleukins), immunocytokines, growth factors, chemoprotectants, vaccines and other biological response modifiers.

Some exemplary embodiments relate to combinations of SAMe with one or more active ingredients that can be prescribed or used for treatment of and/or prophylaxis of a joint or inflammatory disease/disorder in a subject include, but are not limited to, analgesics, non-steroidal anti-inflammatory drug compounds (NSAID), disease-modifying antirheumatic drugs (DMARDs), corticosteroids, anakinra (an interleukin-1 receptor antagonist), COX-2 inhibition, gamma-aminobutyric acid-B (GABAB) receptor agonists, such as baclofen, GABAA potentiating drugs, such as the benzodiazepines tumor necrosis factor (TNF)-inhibiting drugs, and other drugs that modify the immune response (immunosuppressive drugs).

Some exemplary embodiments provided herein relate to combinations of SAMe with one or more active ingredients that can be prescribed or used for treatment of and/or prophylaxis of an autoimmune disease/disorder in a subject include, but are not limited to, DMARDs, corticosteroids, anakinra (an interleukin-1 receptor antagonist), TNF-inhibiting drugs, and other drugs that modify the immune response (immunosuppressive drugs).

Some exemplary embodiments provided herein relate to combinations of SAMe with one or more active ingredients that can be prescribed or used for treatment of and/or prophylaxis of a degenerative disease/disorder in a subject include, but are not limited to, NSAIDs, COX-2 inhibition, GABAB receptor agonists, such as baclofen, and GABAA potentiating drugs, such as the benzodiazepines.

Some exemplary embodiments provided herein relate to combinations of SAMe with one or more active ingredients that can be prescribed or used for treatment of and/or prophylaxis of a soft tissue disease/disorder in a subject include, but are not limited to, milnacipram, pregabalin, SNRIs, NSRIs, muscle relaxers, sedatives, painkillers, and NSAIDs.

Some exemplary embodiments provided herein relate to combinations of SAMe with one or more active ingredients that can be prescribed or used for treatment of and/or prophylaxis of a genetic disease/disorder related to hyper or hypo methylation in a subject include, but are not limited to methionine, MTA (5′-deoxy-5′-(methylthio)adenosine) and other SAMe metabolites.

Some exemplary embodiments provided herein relate to combinations of SAMe with one or more active ingredients that can be prescribed or used for treatment of and/or prophylaxis of a gastrointestinal disease/disorder in a subject include, but are not limited to, 5-Aminosalicylic acid (5-ASA) medications, Corticosteroids (prednisone), immunomodulatory medications such as Azathioprine (Immuran), 6-Mercaptopurine (6-MP), Methotrexate and Cyclosporine (Sandimmune), commonly used antibiotics such as Metronidazole (Flagyl) and Ciprofloxacin (Cipro) and biologic agents such as Infliximab (Remicade).

Some exemplary embodiments provided herein relate to combinations of SAMe with one or more active ingredients that can be prescribed or used for treatment of and/or prophylaxis of a cardiovascular disease/disorder in a subject include, but are not limited to, statins, angiotensin-converting enzyme (ACE) inhibitors, ASA, SAMe break down products such as methionine, MTA and folate, cardioprotectants, vasoprotectants, coagulation inhibitors.

Some exemplary embodiments provided herein relate to combinations of SAMe with one or more active ingredients that can be prescribed or used for the treatment of and/or prophylaxis of a metabolic disease in a subject including, but not limited to sulfonylureas, biguanides, thiazolidinediones, alpha-glucosidase inhibitors, meglitinides, incretin-based therapies, and DPP-4 inhibitors.

Some exemplary embodiments provided herein relate to combinations of SAMe with one or more active ingredients that can be prescribed or used for treatment of and/or prophylaxis of a disorder induced in whole or in part by oxidative or free-radical damage including, but are not limited to, antioxidants such as Vitamin A, Vitamin C, Vitamin E, polyphenols, flavonoids, selenium, carotenoids.

Some exemplary embodiments provided herein relate to combinations of SAMe with one or more active ingredients that can be prescribed or used for treatment of and/or prophylaxis of a disorder induced in whole or in part by damage to the central nervous system such as brain injury or spinal cord injury including, but not limited to, neuroprotectants, nootropic agents, CNS modulators, analgesics, muscle relaxants, apoptosis inhibitors, bone modulators, antioxidants.

Some exemplary embodiments provided herein relate to combinations of SAMe with methionine, MTA, folate, vitamin B6 and/or B12. These agents may be correlated with lowering homocysteine production. Therefore, it is considered that combining SAMe with methionine, MTA, folate, vitamin B6 and/or B12 may result in increased supplementation of SAMe by enhancing the body's natural ability to make SAMe while at the same time supplementing SAMe with exogenous SAMe exhibiting enhanced absorption and improved bioavailability. As used herein the term “folate” refers to vitamin B9 in all of its natural and synthetic forms including, but not limited to, folic acid, tetrahydrofolate and L-methylfolate.

In some embodiments, an exemplary enhanced-absorption SAMe dosage form provided herein may be included in a kit with a separate dosage form containing at least one other active ingredient, exemplified by one or more compounds suitable for the treatment of or commonly prescribed or used for the treating and/or prophylaxis in a subject a disease or disorder selected from the group consisting of, but not limited to, a mental or psychiatric disorder (e.g. psychotic/mood or non-psychotic mental disorders such as depression and substance related disorders, respectively), a nervous system disease/disorder (e.g. a central nervous system disease such as Alzheimer's), other neurological disease/disorders (e.g. headaches and sleep disorders), conditions associated with injury to the central nervous system, a liver disease/disorder (e.g. alcoholic liver disease), a cancer (e.g. solid and blood-borne cancers), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper or hypo methylation, a gastrointestinal disease/disorder, a cardiovascular disease/disorder, and a disorder induced in whole or in part by oxidative or free-radical damage, comprising administering to said subject an exemplary composition provided herein which improves the absorption of a physiologically effective amount of exogenous SAMe.

In addition to combinations of SAMe with the one or more additional ingredients exemplified above or methionine, MTA, folate, vitamin B6 and/or B12, administration of the exemplary enhanced-absorption SAMe formulations provided herein may also augment the effects of other drugs or nutritional supplements being taken by the subject. Thus, some exemplary embodiments provided herein relate to combinations of enhanced-absorption SAMe formulations with drugs or nutritional compounds already employed for treating other diseases for increasing the activity of said drugs or nutritional compounds.

The present embodiments are further described by the following examples. These examples, while illustrating certain specific aspects provided herein, should not be considered to limit or circumscribe the scope of the disclosed embodiments.

### EXAMPLES

### Example 1

### Altered SAMe Coating Compositions Result in GI Segment-Specific SAMe Absorption

In order to better understand the absorption characteristics of SAMe in vivo, standard, uncoated tablets comprising SAMe were first generated and then covered with a segment-specific coating targeting one of three distinct regions of the GI tract.

The uncoated SAMe tablets comprising microcrystalline cellulose, croscarmellose, colloidal silicon dioxide and magnesium stearate were made using standard procedures known to those skilled in these arts. In order to improve the compressibility of the composition, SAMe powder was granulated using a dry compaction process. Each excipient was split between the intra-granular and extra-granular phases. The final tableting mixture was compressed using a rotary tablet press fitted with elongated oval tooling at one station and the remaining stations blocked off. The relative ambient humidity was maintained at around 30% or less and ambient temperature was controlled between 20 and 30° C. throughout the process. The granules used in this formulation had good flow properties and demonstrated no sticking or picking during compression.

In order to target SAMe release in the proximal GI tract (duodenum & jejunum) a commonly used Eudragit® coating known to dissolve above a pH of about 5.5, was applied to the SAMe tablets (EUDRAGIT is a registered trademark of Rohm GmbH; Darmstadt, Germany). In addition, prior to applying the pH-dependent coating, a commercially available seal coat was first applied.

A second formulation comprising a second commercially available Eudragit® coating was utilized to deliver SAMe to the distal GI tract (ileum/ascending colon) as it dissolves at a pH above about 7.0. As above, this formulation was first prepared with a commercially available seal coat.

Finally, a rate controlling coating used to provide metered SAMe release throughout the entire GI tract was applied to uncoated SAMe core tablets by a Contract Research Organization.

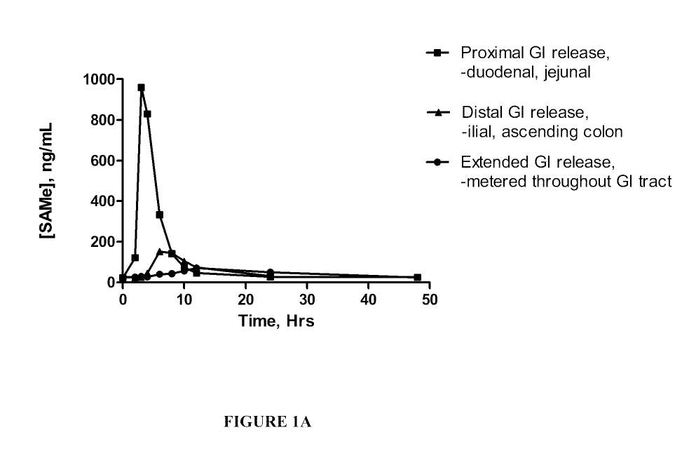

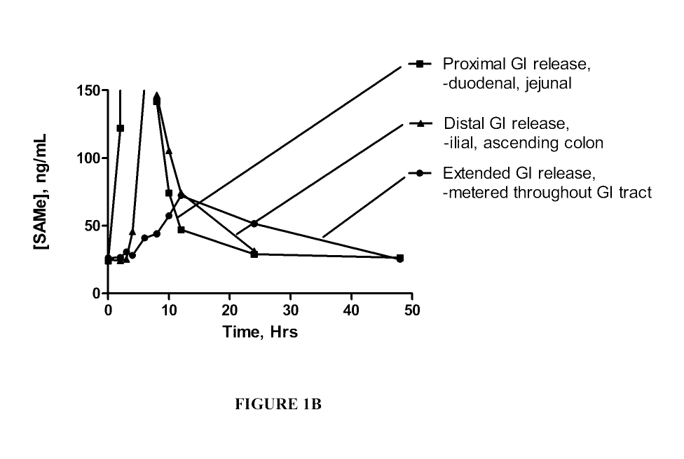

As seen in FIG. 1A, delivery of SAMe is achieved in all three regions of the GI tract and each site results in a unique pharmacokinetic profile of SAMe with different Cmax and Tmax values (FIG. 1B). In the case of the pH 5.5 and pH 7.0 coated tablets, the expected time of un-coating is a function of the pH sensitivity of the coating together with the expected transit or arrival time for the targeted segments. The Tmax observed experimentally corresponded to that anticipated time. In the case of the rate-controlling coating which is a non-dissolving coating, there is no seal coating; the Tmax corresponds to maximal drug delivery in the colon.

The three formulations therefore were confirmed to show targeted delivery at three distinct sites within the GI tract corresponding to proximal, distal and extended GI segments as judged by their Tmax. The core of the three products and the dose applied was identical but the amount delivered as indicated by the Cmax and the AUC was significantly different. The relative bioavailability correlates with the tight junction porosity of the targeted segment.

### Example 2

### In Vivo Delivery and Analysis of Absorption-Enhancing Agents

Use of absorption enhancers as a means to increase the absorption and thus bioavailability of a novel preparation of SAMe is achieved by either co-formulating SAMe with one or more absorption enhancers or co-administering SAMe with one or more absorption-enhancing agents. Co-administration may not necessarily be at the same time as it may be more efficacious to administer said absorption enhancers within a reasonable time either before or after administration of said proprietary preparation of SAMe.

Identification of suitable absorption enhancers may be found in the art or may be achieved in vivo. In vivo activity of compositions comprising SAMe and one or more absorption enhancing agent may be measured after administration into an animal model. Preferably, the animal model comprises a pharmacokinetic (PK) model wherein candidate formulations are administered using pharmacologically effective doses to non-rodent animals (for example dog, pig, mini-pig, or primate) and blood, urine, cerebrospinal fluid (CSF) or other appropriate biological fluid is removed at periodic intervals. The biological fluid is tested for active compound in order to construct concentration vs. time profiles. These data are analyzed and pharmacokinetic parameters are calculated in order to assess in vivo pharmacokinetic activity. The most common pharmacokinetic parameters analyzed in such models are Cmax, Tmax, and area under the curve (AUC).

Alternatively, or in addition to the PK model, one can identify suitable absorption enhancers using efficacy as a measurement, through the use of non-rodent models for liver disease or osteoarthritis as an example.

Plasma and urine markers include measuring markers suitable for each disease/disorder.

Changes in gene expression include serial analysis of gene expression (genomics) and changes in protein expression (proteomics) or changes in metabolite levels (metabolomics).

### Example 3

### In Vitro Screening of Absorption-Enhancing Agents

In addition to above, identification of suitable absorption enhancers may also be achieved using simple, standard in vitro screening assays. In the present embodiment, permeability of SAMe across Caco-2 cell monolayers treated with an absorption enhancer is used to identify agents which increase the amount of SAMe absorbed by said Caco-2 cells in comparison to untreated Caco-2 cell monolayers. The Caco-2 cell line is derived from a human colorectal carcinoma and is widely used for in vitro cell culture models for the study of gastrointestinal drug absorption (Stewart, B., (1995) Pharm. Res. 12:693). In these models, pure cell lines are grown on a semi-permeable membrane. Drug formulations are placed on the apical or basolateral side of the cell monolayer and transport is determined via measurement of drug concentrations on the other side of the membrane.

The Caco-2 cell line utilized here was from the American Type Culture Collection (ATCC). Caco-2 cells are grown in Dulbecco's modified Eagle's medium (DMEM, Gibco) supplemented with 20% FBS (fetal bovine serum, Gibco), 100 uM non-essential amino acids (NEAA, Gibco) and 2 mM L-glutamine (Gibco). A Beckton Dickinson BIOCOAT® HTS Caco-2 Assay System Kit is used resulting in 6.6×10⁵ cells/cm² seeding density (BIOCOAT is a registered trademark of Collaborative Biomedical Products, Inc., Bedford, Mass., USA). The cells used in transport studies are grown for 3 days before the experiments. The culturing conditions are 37° C. in an atmosphere of 5% CO₂ and 100% humidity.

For permeability across Caco-2 cell monolayers, the transport medium used was Hank's Buffered Salt Solution (HBSS; purchased from Gibco) containing D-glucose, and HEPES pH adjusted to 7.4. A 2 mM aqueous solution of either SAMe tosylate disulfate or SAMe 1,4 butanedisulfonate was added on the apical or basolateral side according to the manufacturer's procedure for the Caco-2 kit. Samples were measured after 120 minute incubation by liquid chromatography-mass spectrometry (LC/MS). The integrity of the monolayers was monitored using Lucifer Yellow Assay. As an example, the effect of the absorption enhancer (and specifically a tight junction opening agent), EDTA (2 mM in wells), as well as calcium-free medium on Caco-2 permeability of SAMe is compared to absorption of SAMe on its own. Propranolol is a high permeability marker and was utilized as a positive control for a readily absorbed molecule.

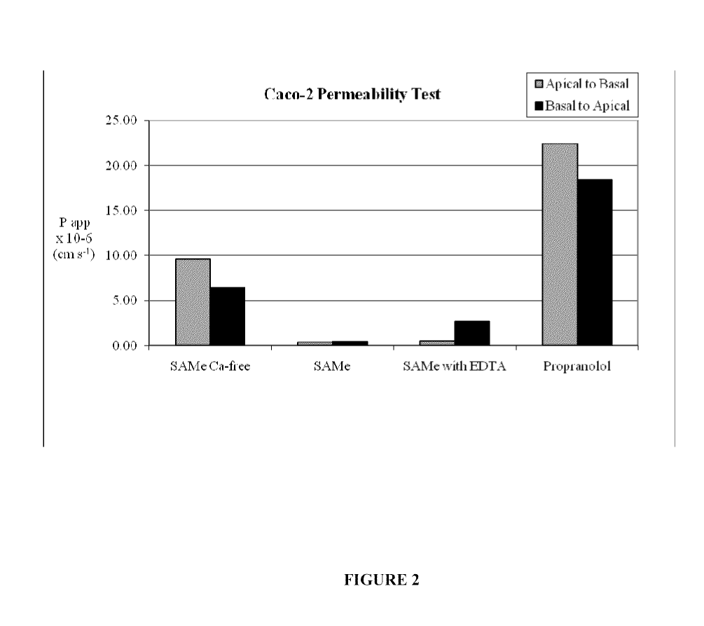

The results in Table 2 below as well as those depicted in FIG. 2 show that SAMe absorption on its own is low for both salts as evidenced by a low apparent permeability coefficients (Papp=0.41×10⁻⁶ and 0.50×10⁻⁶ cm s⁻¹ in apical to basolateral and basolateral to apical directions, respectively for SAMe disulfate tosylate; and Papp=0.50×10⁻⁶ and 0.60×10⁻⁶ cm s⁻¹ in apical to basolateral and basolateral to apical directions, respectively for SAMe 1,4 butanedisulfonate). Interestingly, the two stable salts of SAMe, disulfate-tosylate and 1,4-butanedisulfonate, had identical permeability profiles within experimental error providing the evidence of their biological equivalence. The remaining permeability studies were carried out with SAMe disulfate-tosylate.

The measured permeability values of the SAMe salts were much lower than those measured with the high permeability marker, propranolol (22.4×10⁻⁶ and 18.4×10⁻⁶ cms⁻1, respectively.) Permeability coefficients that are concentration independent and/or similar in apical to basolateral as well as basolateral to apical directions are said to be characteristic for paracellular transport. Paracellular transport mechanism for SAMe was supported here by a 13-24 fold increase in SAMe permeability in the calcium-free buffer as well as a 1.3-5.0 fold increase when in the presence of a known tight junction opener, EDTA (as shown in Table 2 and FIG. 2).

TABLE 2
Permeability of SAMe Across a Monolayer of Caco-2 Cells

| | Apical to | | Basal to | |
|---|---|---|---|---|
| | Basal × 10⁻⁶ cm s⁻¹ | | Apical × 10⁻⁶ cm s⁻¹ | |
| Well Contents | Papp_a-b | Papp_a-b SD | Papp_b-a | Papp_b-a SD |
| SAMe 1,4 butanedisulfonate | 0.50 | 0.08 | 0.60 | 0.13 |
| SAMe disulfate tosylate | 0.41 | 0.04 | 0.50 | 0.03 |
| SAMe disulfate tosylate | 9.60 | 0.56 | 6.48 | 0.75 |
| Calcium-free | | | | |
| SAMe disulfate tosylate with | 0.54 | 0.04 | 2.63 | 0.16 |
| EDTA | | | | |
| Propranolol (Control) | 22.44 | 1.73 | 18.36 | 0.34 |

### Example 3a

### SAMe Permeability is Increased Both In Vitro and In Vivo in the Presence of Tight Junction Modulators

Additional Caco-2 testing was performed on a number of tight junction opening agents as well as in vivo studies on two candidate tight junction opening agents at a contract research organization. Similar to the description above, the Caco-2 cell line was obtained from ATCC and grown in DMEM (Sigma-Aldrich) supplemented with 20% FBS (Sigma-Aldrich), 100 uM non-essential amino acids (Sigma-Aldrich) and 2 mM L-glutamine (Sigma-Aldrich). The Caco-2 cells grown in tissue culture flasks were trypsinized, suspended in medium, and the suspensions were applied to wells of a collagen-coated BioCoat Cell Environment in 24-well format at 24,500 cells per well. The cells were allowed to grow and differentiate for three weeks, feeding at 2-day intervals.

For apical to basolateral permeability, a 2 mM aqueous solution of SAMe tosylate disulfate was added to the apical side and the amount of permeation was determined on the basolateral side. The apical and basolateral side buffers contained modified Transport Buffer (25 mM HEPES, 1× Hank's Balanced Salt Solution (Sigm-Aldrich)) pH 7.4. Caco-2 cells were incubated with these buffers for 2 hours, and the receiver side buffer was removed for analysis by LC/MS/MS. The receiver, donor, and dosing solution were diluted with an equal volume of 0.2 N HCl immediately after the assay in order to increase SAMe stability. Donor and dosing solution were diluted 100-fold to ensure that the concentration was within the linear range of the assay.

To confirm the integrity of the Caco-2 cell monolayers, TEER (Trans Epithelial Electrical Resistance) measurements were performed on each well at the end of the experiment.

The permeability (Papp) of SAMe is calculated using the following formula:

$$Papp = \frac{d Q}{\frac{d t}{CoA}}$$

Where dQ/dt is the rate of permeation, C₀ is the initial concentration of test agent, and A is the area of the monolayer.

As shown in Table 3 below, the presence of two different fatty acids, either a C10 fatty acid or a sulfonic acid, resulted in a dramatic increase in the permeability of SAMe.

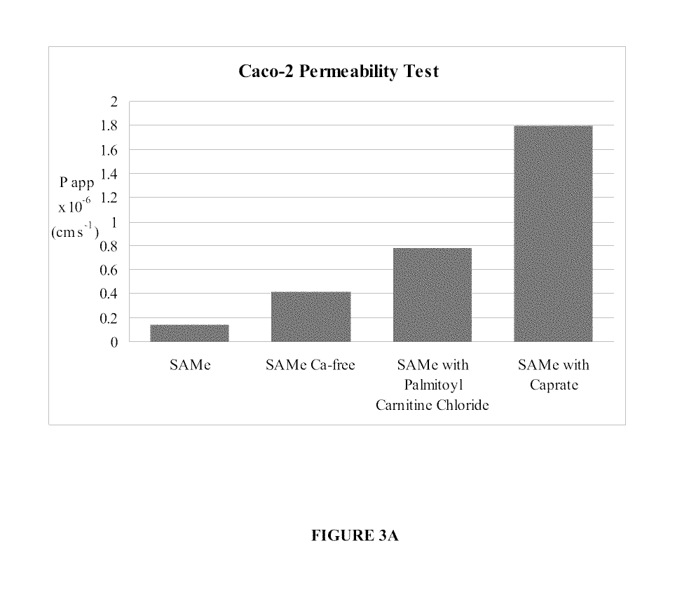

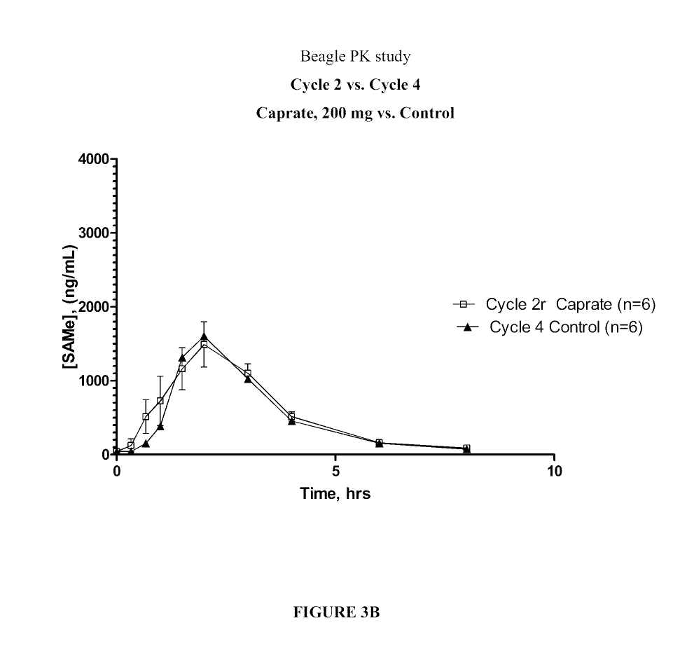

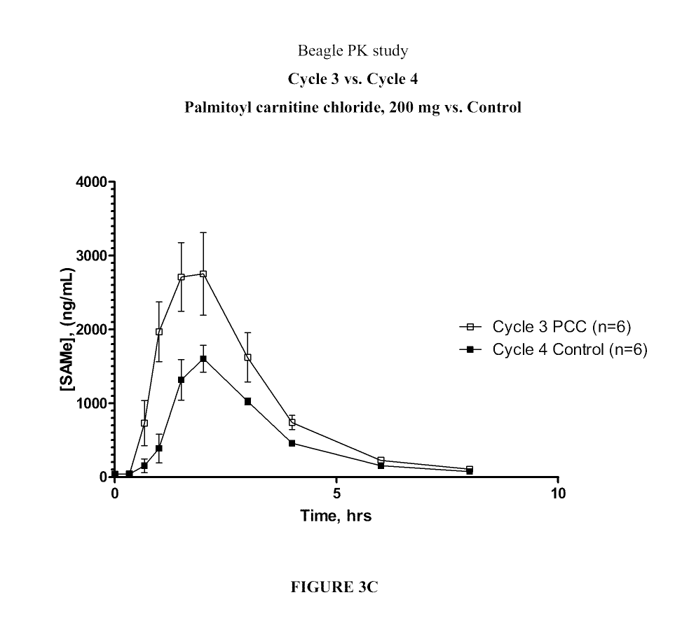

The zwitterionic surfactants, 3-(N,N-Dimethylpalmitylammonio)propanesulfonate and palmitoyl carnitine chloride were also each tested for their effect on SAMe uptake and both showed marked increase in SAMe permeability as seen in Table 3. In addition, alpha-cyclodextrin and a dicarboxcylic acid each resulted in a 5-6 fold increase in SAMe permeability. The low and high permeability controls, ranitidine and warfarin, respectively, verify the use of these absorption enhancing agents as an in vitro screening method for measuring the effect of these agents on SAMe permeability across Caco-2 monolayers. Furthermore, the permeability of SAMe alone, versus SAMe in the absence of calcium or in the presence of the tight junction modulators, palmitoyl carnitine chloride or caprate is depicted in the graph in FIG. 3A.

TABLE 3
Permeability of SAMe in the Presence of Various Tight Junction Modulators

| | | | | | | Mean | |
|---|---|---|---|---|---|---|---|
| | | | | Permeability | | TEER | Fold |
| | Chemical | Caco-2 | | Coefficient | | Resistance | Transport |
| Test Article | Class | Concentration | | (×10⁻⁶ cm/s) | | (ohms) | Increase |
| SAMe | API control | 2 | mM | 0.14 | (run 2) | 782 | 1X |
| (control) | | | | 1.6 | (run 3) | 901 | 1X |
| | | | | 1.6 | (run 4) | 1004 | 1X |
| Sodium Caprate | Fatty acid | 14 | mM | 1.79 | (run 2) | 235 | 12.8X |
| | | | | 9.1 | (run 4) | 260 | 5.7X |
| | | 7 | mM | 4.1 | (run 4) | 905 | 2.6X |
| Sodium 1- | Fatty acid | 14 | mM | 3.1 | (run 2) | 120 | 21.9X |
| decanesulfonate | | | | 15.5 | (run 3) | 135 | 9.7X |
| | | 7 | mM | 6.3 | (run 3) | 165 | 3.9X |
| Palmityldimethyl | Zwitterionic | 0.6 | mM | 3.8 | (run 4) | 415 | 2.4X |
| ammonio propane | surfactant | 0.3 | mM | 6.0 | (run 3) | 420 | 3.8X |
| sulfonate | | | | 1.4 | (run 4) | 434 | 0.9X |
| Palmitoyl | Zwitterionic | 0.15 | mM | 0.78 | (run 2) | 272 | 5.6X |
| Carnitine | surfactant | | | 5.0 | (run 4) | 519 | 3.1X |
| Chloride (PCC) | | 0.4 | mM | 10.0 | (run 4) | 366 | 6.3X |
| Dodecyltrimethyl | Fatty amine | 14 | mM | 10.5 | (run 4) | 431 | 6.6X |
| Ammonium | | | | | | | |
| Bromide | | | | | | | |
| Alpha- | Cyclodextrin | 51.4 | mM | 0.75 | (run 2) | 293 | 5.4X |
| Cyclodextrin | | | | 6.3 | (run 4) | 341 | 3.9X |
| Dicarboxylic acid | Organic acid | 66.6 | mM | 10.5 | (run 3) | 143 | 6.6X |
| | | | | 15.5 | (run 4) | 136 | 9.7X |
| Ranitidine | Low | 50 | uM | 0.91 | (run 2) | 1080 | n/a |
| | permeability | | | 0.91 | (run 3) | 975 | n/a |
| | control | | | 1.0 | (run 4) | 931 | n/a |
| Warfarin | High | 50 | uM | 48.8 | (run 2) | 976 | n/a |
| | permeability | | | 49.0 | (run 3) | 941 | n/a |
| | control | | | 46.6 | (run 4) | 1011 | n/a |

To further investigate the effects of these tight junction modulators on SAMe absorption, in vivo pharmacokinetic testing of caprate and PCC were performed using animal subjects. Caprate is one of the most well-recognized tight junction modulators used in pharmaceutical development. Formulations comprising caprate have advanced through to Phase III clinical trials demonstrating its effectiveness and safety. For this reason, formulations comprising SAMe as well as caprate were first investigated for pharmacokinetic analysis in vivo.

Fasted male beagle dogs (7-10 kg) were used to conduct a single dose, crossover study to compare the bioavailability of SAMe+caprate enhancer with SAMe control capsules (Table 4). The study protocol was approved by the institution's Animal Care Committee, and all animals were cared for according to regulations proposed by Agriculture Canada and the USDA. Each group (6 dogs for SAMe/caprate and 6 dogs for the control) was dosed with a single orally administered enteric coated capsule, under fasted conditions, followed by 5 mL of purified water orally with a syringe to facilitate swallowing. Pharmacokinetic blood samples (2 mL each) for SAMe analysis were collected from the jugular vein using the following timepoints: pre-dose, 20 and 40 minutes, 1, 1.5, 2, 3, 4, 6, and 8 hours after treatment. The venipuncture blood samples were collected into tubes containing the anticoagulant K2-EDTA, and stored on wet ice pending processing. Following collection, samples were centrifuged (at 4° C.) to separate the plasma fraction from the blood cells. The resulting plasma fraction was recovered and stored frozen (at −80° C.) using polypropylene tubes pending bioanalytical analysis.

The concentration of SAMe in dog plasma was determined using a well established LC/MS/MS method. This method employs stable-isotope dilution liquid chromatography-electrospray injection tandem mass spectrometry (LC-ESI-MS/MS) to determine SAMe and SAH in plasma. The analysis used to calculate the main pharmacokinetic parameters (Cmax, Tₘₐₓ, and AUC) was conducted using GraphPad Prism® 5 software.

The capsules tested in beagle dogs were formulated using size #13 gelatin capsules, each containing 200 mg SAMe ion. The capsules contained varying amounts of additional excipients (Microcrystalline cellulose, Sodium Starch Glycolate, Colloidal Silicon Dioxide, and Magnesium Stearate), with and without 200 mg of caprate enhancer. Capsules were manually filled and the resulting formulations are summarized in Table 4:

TABLE 4
Capsule formulations of SAMe with and without Sodium Caprate

| | Control capsule | Caprate capsule |
|---|---|---|
| Component | (mg/capsule) | (mg/capsule) |
| Capsule Core | | |
| SAMe disulfate tosylate | 384.5 | 384.5 |
| Microcrystalline Cellulose 113 | 79.3 | 79.3 |
| Microcrystalline Cellulose 112 | 803.1 | 803.1 |
| Sodium Starch Glycolate Type A | 27.5 | 27.5 |
| Colloidal Silicon Dioxide | 2.8 | 2.8 |
| Magnesium Stearate | 5.5 | 5.5 |
| Sodium Caprate | 0 | 200 |
| | | (representing |
| | | 176 mg of caprate) |

Prior to applying an enteric coating to the capsules, a seal coat was first applied in order to improve the capsule surface properties. A stirred 12% suspension of a commercially available seal coat in purified water was applied to the uncoated SAMe capsules in an Aeromatic Coating Column using 55° C. inlet air temperature and 4-6 g/min spray rate until 4% weight gain was achieved (Table 5). A plasticized 80% solid (w/w) commercially available enteric coat designed to dissolve at pH 5.5 was then applied to the tablet formulations such that they would remain intact within the stomach. The enteric coating was generated using a stirred 30% aqueous suspension (56% of final coating suspension by weight), plasticizer 20% aqueous suspension (7% of final coating suspension by weight), triethyl citrate (2% of final coating suspension by weight) and purified water (35% of final coating suspension by weight). These components were mixed and then applied to the seal coated SAMe capsules in an Aeromatic Coating Column using 55° C. inlet air temperature and 4-6 g/min spray rate, until a 6% weight gain was achieved (Table 5).

TABLE 5
Coating composition for capsules

| | Seal Coat | Control capsule | Caprate capsule |
|---|---|---|---|
| | Composition | (% weight | (% weight |
| Component | (% of Solids) | of capsule) | of capsule) |
| Seal Coat | | | |
| LustreClear LC103 | 93% | 4% | 4% |
| Ferric Oxide | 0.7% | | |
| Titanium Dioxide | 3.3% | | |
| Simethicone | 2.8% | | |
| Enteric Coat | | | |
| Eudragit L30 D55 | 86.8% | 6% | 6% |
| Triethyl Citrate | 10.3% | | |
| Plasacryl T20 | 2.9% | | |

Surprisingly and quite unexpectedly, the presence of caprate did not enhance the in vivo bioavailability of SAMe as shown in FIG. 3B. The control Cmax was 1627 ng/mL compared to a Cmax of 1597 ng/mL for the caprate formulations and the AUC values were 4191 hg hr/mL (control) versus 4510 ng hr/mL (with caprate). Even more surprising is the data in FIG. 3C, which shows the effect of the zwitterionic surfactant, palmitoylcarnitine chloride (PCC), on SAMe bioavailability. Experiments were carried out as described above for caprate except that PCC was substituted for formulations in place of caprate.

Capsules comprising SAMe and 200 mg of DL-palmitoylcarnitine chloride (representing 183.4 mg of the DL-palmitoylcarnitine ion) were administered to 6 male beagle dogs as above and the amount of SAMe in the plasma was compared to the control group of 6 male beagle dogs administered a single dose of the control (SAMe alone). The composition of the PCC formulation is set forth in Table 6.

TABLE 6
Capsule formulations of SAMe with and without PCC

| | Control capsule | PCC capsule |
|---|---|---|
| Component | (mg/capsule) | (mg/capsule) |
| Capsule Core | | |
| SAMe disulfate tosylate | 384.5 | 384.5 |
| Microcrystalline Cellulose 113 | 79.3 | 79.3 |
| Microcrystalline Cellulose 112 | 803.1 | 803.1 |
| Sodium Starch Glycolate Type A | 27.5 | 27.5 |
| Colloidal Silicon Dioxide | 2.8 | 2.8 |
| Magnesium Stearate | 5.5 | 5.5 |
| DL-palmitoylcarnitine chloride (PCC) | 0 | 200 |
| | | (representing |
| | | 183.4 mg |
| | | of DL-palmitoyl- |
| | | carnitine ion) |

Surprisingly, the presence of PCC resulted in a significant increase in the amount of SAMe present in the plasma as compared to the control subjects (FIG. 3C). The SAMe Cmax in the presence of PCC was approximately 91% higher than the Cmax of the control group (3108 ng/mL and 1627 ng/mL, respectively). In addition, the SAMe AUC increased by approximately 86% in the presence of PCC (7794 ng hr/mL compared to 4191 ng hr/mL for the control).

This data, together with the in vitro screening results in Table 3 above, show for the first time that a subset of high potency tight junction opening absorption enhancers exists and that identification of potential high potency tight junction opening agents may be carried out using, for example, a Caco-2 cell model as described above. Based on the concentration of tight junction modulator utilized as well as the increase in SAMe permeability achieved, suitable high potency tight junction modulators elicit at least a two-fold increase in SAMe uptake in vitro when used at low concentrations (i.e. about 2 mM or below). More preferably, at least a three-fold increase in SAMe uptake occurs at low concentrations of these tight junction opening agents and, even more preferably, a four-fold or greater increase is achieved.

### Example 3b

### The Effect of Additional Zwitterionic Surfactants on SAMe Permeability and Bioavailability

In vitro and in vivo experiments are carried out as described in Example 3a using acyl carnitines with either longer or shorter chain length in comparison to PCC (see previous Table detailing acyl carnitines). For example, Caco-2 cell and beagle experiments as described above are conducted using stearoyl carnitine, myristoyl carnitine, lauroyl carnitine, decanoyl carnitine or mixtures of two or more thereof.

Experiments described here and above may also be carried out using other solid or semi-solid, oral dosage forms, such as tablets. Tablets are typically generated as described in Example 1 as well as in co-owned and co-pending U.S. patent application Ser. No. 12/845,600.

### Example 3c

### SAMe Permeability and Bioavailability Increase in the Presence of Various Sulfobetaines

In vitro and in vivo experiments measuring SAMe permeability and pharmacokinetic parameters, respectively, as described in Example 3a were performed using dimethylammoniopropane sulfonates sulfobetaines) of varying chain length (carbon chain length of 10-18 as listed below).

In vitro Caco-2 testing was performed as detailed above using 0.15 mM of sulfobetaine-10, sulfobetaine-12, sulfobetaine-14, sulfobetaine-16, or sulfobetaine-18 as the enhancer. The presence of sulfobetaine-10 or sulfobetaine-12 did not improve SAMe permeability across the Caco-2 cell monolayer. However, as shown in Table 7 below, the presence of sulfobetaine-14, sulfobetaine-16 or sulfobetaine-18 at a concentration of 0.15 mM resulted in at least a five-fold increase in SAMe permeability across the Caco-2 cell monolayer, and permeability increased with increasing acyl chain length. These three sulfobetaines were thus identified as high potency absorption enhancers as defined in the present application.

As part of these in vitro studies, the degree of Caco-2 cell recovery (as observed by degree of permeability and cell attachment 24 hours post treatment with each tight junction modulator) was also investigated. This 24 hour resistance recovery test is an indirect measure of epithelial cell disruption and data supporting a trend toward less in vitro disruption may be associated with less impact in vivo. The results in Table 7 show that cell recovery improves with decreasing sulfobetaine chain length.

TABLE 7
Permeability of SAMe in the Presence of Various Sulfobetaines

| | | | | | TEER |
|---|---|---|---|---|---|
| | | | | Mean | Resist. |
| | | Permeability | Fold | TEER | 24 hour |
| | Caco-2 | Coefficient | Transport | Resistance | post assay |
| Enhancer | Conc. | (×10⁻⁶ cm/s) | Increase | (ohms) | (ohms) |
| SAMe | 2 mM | 9.6 | 1X | 462 | |
| control | | 0.14 | 1X | 782 | |
| | | 1.6 | 1X | 901 | |
| | | 1.6 | 1X | 1004 | |
| | | 0.14 | 1X | 1045 | 2280 |
| | | 0.17 | 1X | 1031 | 2181 |
| | | 0.26 | 1X | 726 | 1440 |
| SAMe | 2 mM | 0.186 | 1X | 924 | 1975 |
| control | | 0.131 | 1X | 1285 | 1871 |
| | | 0.124 | 1X | 995 | 1740 |
| Sulfobe- | 0.15 mM | 0.218 | 1.2X | 1043 | 1553 |
| taine-8 | | | | | |
| Sulfobe- | 0.15 mM | 0.257 | 1.4X | 934 | 1923 |
| taine-12 | | | | | |
| Sulfobe- | 0.15 mM | 0.654 | 5.0X | 263 | 1892 |
| taine-14 | | | | | |
| Sulfobe- | 0.6 mM | 3.8 | 2.4X | 415 | |
| taine-16 | | | | | |
| (Palmityl- | | 7.4 | 51.9X | 25 | 33 |
| dimethyl | 0.3 mM | 6.0 | 3.8X | 420 | |
| ammonio | | 1.4 | 0.9X | 434 | |
| propane) | | 2.5 | 17.7X | 41 | 84 |
| sulfonate | 0.3 mM | 2.19 | 17.7X | 71 | 30 |
| | 0.15 mM | 1.9 | 13.3X | 99 | 1602 |
| | | 0.678 | 5.2X | 123 | 292 |
| Sulfobe- | 0.15 mM | 0.649 | 5.0X | 123 | 292 |
| taine-18 | | | | | |

To further analyze the effects of these tight junction modulators, in vivo experiments using fasted male Beagle dogs as detailed in Example 3a were carried out using SAMe capsules comprising either sulfobetaine-14 or sulfobetaine-16. Capsules comprising 200 mg SAMe (ion) and 200 mg of sulfobetaine-14 or sulfobetaine-16 were administered to six male beagle dogs as above and the amount of SAMe in the plasma was compared to the control group of six male beagle dogs administered a single dose of the control (200 SAMe (ion) alone). The compositions of the sulfobetaine and control formulations are set forth below in Table 8.

TABLE 8
Capsule formulations of SAMe with and without
Sulfobetaine-14 or Sulfobetaine-16

| | Control capsule | Sulfobetaine capsule |
|---|---|---|
| Component | (mg/capsule) | (mg/capsule) |
| Capsule Core | | |
| SAMe disulfate tosylate | 384.5¹ | 384.5¹ |
| Microcrystalline Cellulose 113 | 79.3 | 79.3 |
| Microcrystalline Cellulose 112 | 803.1 | 803.1 |
| Sodium Starch Glycolate Type A | 27.5 | 27.5 |
| Colloidal Silicon Dioxide | 2.8 | 2.8 |
| Magnesium Stearate | 5.5 | 5.5 |
| Sulfobetaine-14 or Sulfobetaine-16 | 0 | 200 |

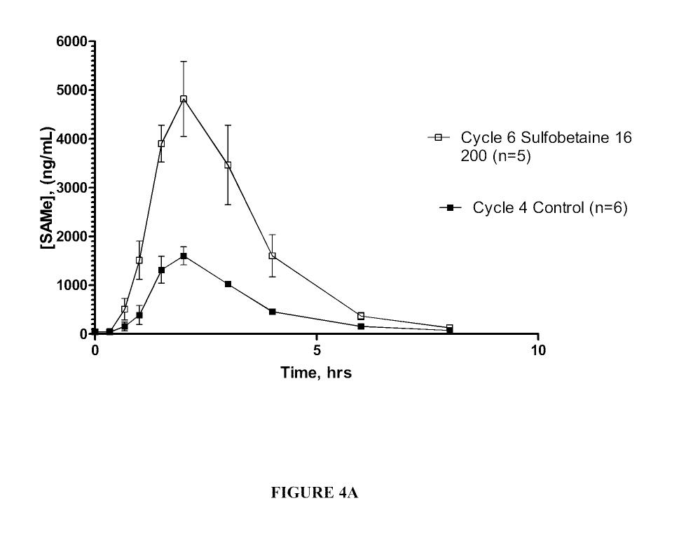

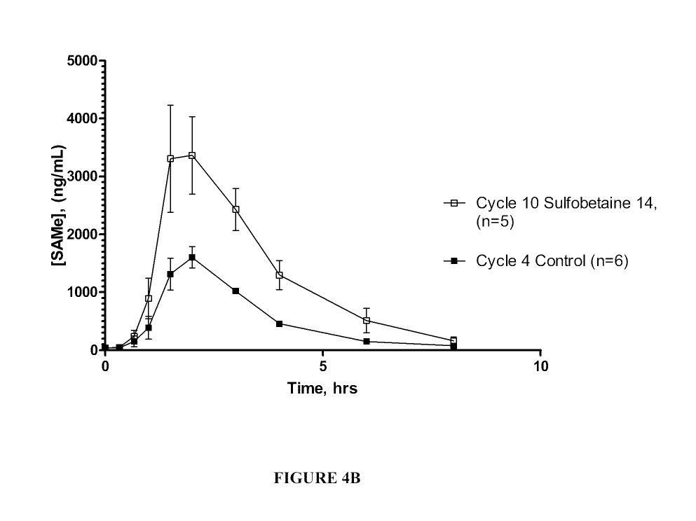

As identified in FIG. 4A, the presence of sulfobetaine-16 resulted in a significant increase in the amount of SAMe present in the plasma as compared to the control subjects. As shown, the SAMe Cmax in the presence of sulfobetaine-16 was approximately 316% higher than the Cmax of the control group. In addition, the SAMe AUC increased by approximately 313% in the presence of sulfobetaine-16. Surprisingly, the presence of sulfobetaine-14 also resulted in a significant increase in the bioavailability of SAMe as measured by an increase in the average SAMe Cmax from 1627 ng/mL for the control to 4148 ng/mL in the presence of sulfobetaine-14 (FIG. 4B). The average SAMe AUC also increased from 4191 ng·hr/mL (control group) to 10226 ng·hr/mL in the presence of sulfobetaine-14.

This data supports the finding that a subset of high potency tight junction opening absorption enhancers exists and that identification of suitable high potency tight junction opening agents for increasing SAMe bioavailability in vivo may be carried out using, for example, a Caco-2 cell model as described above.

### Example 4

### Plasma Levels of SAMe Metabolites are not Elevated Relative to Plasma Levels of SAMe

The data presented above clearly demonstrates for the first time that the use of absorption enhancers to increase SAMe permeability in vivo leads to enhanced SAMe bioavailability. This finding is in contrast to the general dogma surrounding SAMe metabolism which continually reports that low SAMe bioavailability is due to active metabolism by the liver upon administration. If this were the case it would be reasonable to believe that the plasma level of one or more SAMe metabolites would be significantly elevated after administration. In order to test this theory, the inventors measured the level of S-adenosyl homocysteine (SAH), the primary metabolite of SAMe, at various time points after administration of a 1600 mg dose of a commercially available SAMe formulation.

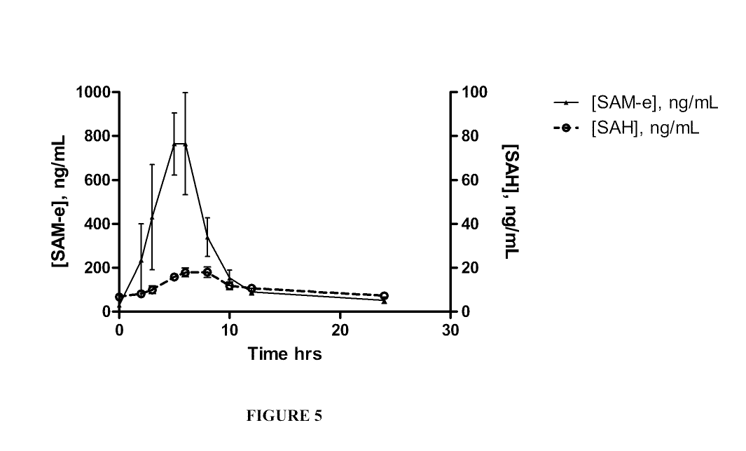

As seen in FIG. 5, at all time points measured, the plasma concentration of SAH (which is plotted on a scale that is tenfold lower than the SAMe concentration) is significantly lower than the level of SAMe itself. Additional SAMe metabolites were also measured and, similarly to SAH, there were no differences in their baseline levels (results not shown.)

These results demonstrate that SAMe metabolism is not responsible for the low bioavailability of SAMe upon administration.

### Example 5

### SAMe Delivered to the Stomach can be Absorbed into the Plasma

Those skilled in these arts are also of the view that SAMe delivery must bypass the stomach in order to avoid its harsh low-pH environment which is taught to cause degradation. In order to even further understand the mechanism of SAMe absorption in vivo, and thus better control its bioavailability, the release of SAMe into the stomach environment and its absorption there from was also assessed.

Uncoated SAMe was formulated into tablets comprising microcrystalline cellulose, croscarmellose, colloidal silicon dioxide and magnesium stearate using standard procedures as described in Example 1.

The absence of an enteric coating in this formulation was intended to cause release of SAMe within the stomach. The resulting pharmacokinetic profile was studied by measuring the presence of the drug in plasma at various time points after administration. A single dose of 400 mg SAMe was given to seven healthy and fasted, male volunteers.

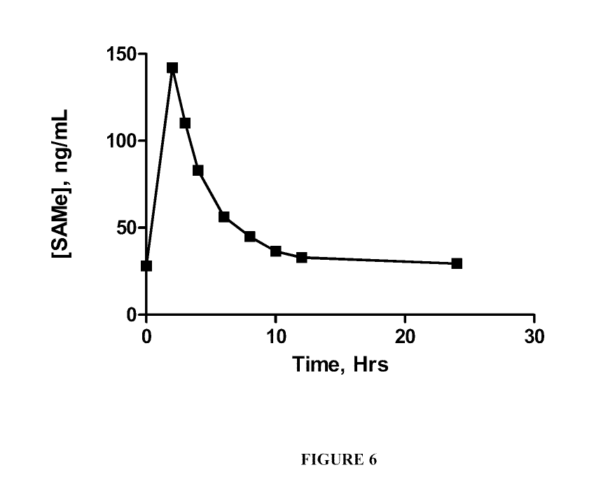

As seen in FIG. 6, the average Cmax of the seven subjects administered the uncoated SAMe formulation was about 145 ng/mL for the 400 mg dose. These results show that, contrary to what is repeatedly reported in the art, SAMe can be delivered to the stomach without using enteric coated formulations and still give rise to increased absorption as seen with the plasma SAMe levels reported here.

1.-103. (canceled)

104. A pharmaceutical composition comprising a therapeutically effective dosage of S-adenosylmethionine and at least one zwitterionic surfactant.

105. The composition of claim 104, wherein said zwitterionic surfactant enhances systemic delivery of S-adenosylmethionine as measured by an increase in Cₘₐₓ and/or AUC compared to a control composition that lacks said zwitterionic surfactant.

106. The composition of claim 105, wherein the Cₘₐₓ and/or AUC of said composition is at least 140% of the Cₘₐₓ and/or AUC obtained with a control composition that lacks said zwitterionic surfactant.

107. The composition of claim 104, wherein said composition is a non-parenteral composition.

108. The composition of claim 107, wherein the non-parenteral composition is an oral dosage composition.

109. The composition of claim 108, wherein the oral dosage composition is formulated as a tablet, a capsule, or a multiparticulate formulation.

110. The composition of claim 108, wherein the oral dosage composition comprises about 50 to about 500 mg of S-adenosylmethionine.

111. The composition of claim 107, wherein the non-parenteral composition is formulated as a dietary supplement or a medical food.

112. The composition of claim 107, wherein at least a portion of the non-parenteral composition is formulated to dissolve in at least one of the stomach, duodenum, jejunum, ileum, large intestine, or colon.

113. The composition of claim 107, wherein the non-parenteral composition comprises a pH sensitive coating.

114. The composition of claim 104, wherein said zwitterionic surfactant is an acyl carnitine.

115. The composition of claim 114, wherein said acyl carnitine is a palmitoyl carnitine, lauroyl carnitine, stearoyl carnitine, myristoyl carnitine, decanoyl carnitine, or a salt thereof.

116. The composition of claim 104, wherein said zwitterionic surfactant is a sulfobetaine.

117. The composition of claim 116, wherein said sulfobetaine is sulfobetaine-10, sulfobetaine-12, sulfobetaine-14, sulfobetaine-16, or sulfobetaine-18.

118. The composition of claim 104, wherein said zwitterionic surfactant is a high potency absorption enhancer as determined in a Caco-2 monolayer permeability assay.

119. The composition of claim 104, wherein S-adenosylmethionine and said zwitterionic surfactant are in a ratio of from 1:100,000 to 100,000:1; 1:150 to 150:1; or 1:10 to 10:1 (weight:weight).

120. The composition of claim 104, wherein S-adenosylmethionine and said zwitterionic surfactant are in a ratio of 1:1 (weight:weight).

121. The composition of claim 104, wherein the composition comprises 50 to 3200 mg of S-adenosylmethionine.

122. The composition of claim 104, further comprising at least one delayed release component.

123. The composition of claim 122, wherein the delayed release component is a pH-triggered enteric coating.

124. The composition of claim 122, wherein the delayed release component is formulated to dissolve in at least one of the duodenum, jejunum, ileum, large intestine, or colon.

125. A method for increasing the bioavailability of exogenous SAMe administered to a subject, said method comprising administering to the subject a non-parenteral composition comprising a therapeutically effective dosage of the composition of claim 104.

126. The method of claim 125, wherein the composition is an oral dosage composition.

127. The method of claim 125, wherein the composition is formulated as a dietary supplement or a medical food.

128. The method of claim 125, wherein at least a portion of the composition is formulated to dissolve in at least one of the stomach, duodenum, jejunum, ileum, large intestine, or colon.

129. The method of claim 128, wherein the composition comprises a pH sensitive coating.

130. The method of claim 125, wherein said zwitterionic surfactant is administered either before or after administration of the composition comprising the at least one therapeutically effective dosage of S-adenosylmethionine.

131. A method of treating a disorder selected from the group consisting of a mental or psychiatric disorder, a nervous system disease or disorder, a neurological disease or disorders, a condition associated with injury to the central nervous system, a liver disease or disorder, a cancer, a joint disease or disorder, an inflammatory disease or disorder, an autoimmune disease or disorder, a degenerative disease or disorder, a soft-tissue disease or disorder, a pain disease or disorder, a genetic disorder related to hyper- or hypo-methylation, a gastrointestinal disease or disorder, a cardiovascular disease or disorder, and a disorder induced in whole or in part by oxidative or free-radical damage, comprising administering to a patient in need thereof a composition of claim 104.

132. The method of claim 131, wherein:
(i) the mental or psychiatric disorder is selected from the group consisting of an anxiety disorder, schizophrenia, major depressive disorder, multi-infarct dementia, minor depression, postpartum depression, inflammatory depression, late-life depression, Parkinson's depression, HIV-associated depression, and bipolar disorder;
(ii) the inflammatory disease or disorder is selected from the group consisting of systemic lupus, inflammatory bowel disease, allergic rhinitis, contact dermatitis, asthma, autoimmune hepatitis, and pelvic inflammatory disease;
(iii) the cardiovascular disease or disorder is selected from the group consisting of hyper- or hypo-homocysteinemia, coronary heart disease, stroke, peripheral vascular disease, and atherosclerotic disease;
(iv) the depressive disorder is a comorbid depression arising in a subject who is or has been undergoing treatment for one or more diseases or disorders selected from the group consisting of cancer, Parkinson's and HIV;
(v) the nervous system disease or disorder or injury is selected from the group consisting of Parkinson's disease, Alzheimer's disease, and cognitive impairment;
(vi) the liver disease or disorder is selected from the group consisting of alcoholic liver disease, non-alcoholic fatty liver disease, viral or non-viral hepatitis, liver cancer, oxidative liver disease, drug induced liver injury, cholestasis, and cirrhosis;
(vii) the cancer is selected from the group consisting of liver cancer, colon cancer, rectal cancer, stomach cancer, esophageal cancer, and adenocarcinoma;
(viii) the joint disease or disorder is arthritis or osteoarthritis;
(ix) the soft-tissue disease or disorder is fibromyalgia;
(x) the pain disease or disorder is selected from the group consisting of fibromyalgia, and abdominal pain; or
(xi) the genetic disorder related to hyper- or hypo-methylation is methylenetetrahydrofolate reductase deficiency.
